(19) United States
(12) Patent Application Publication
(10) Pub. No.: US 2006/0123499 A1
(43) Pub. Date: Jun. 8, 2006

(54) Civet animal model system for Severe Acute Respiratory Syndrome (SARS) coronavirus infection and uses thereof

(76) Inventors: Donglai Wu, Harbin (CN); Xiangang Kong, Harbin (CN); Qingwen Meng, Harbin (CN); Yonggang Liu, Harbin (CN); Yuntao Guan, Harbin (CN); Xunnan Yin, Harbin (CN); Mouping Wang, Harbin (CN); Changwen Li, Harbin (CN); Ming Liao, Guangzhou (CN); Chao-an Xin, Guangzhou (CN); Jinding Chen, Guangzhou (CN); Changchun Tu, Changchun (CN); Hua Xuan, Changchun (CN); Yedong Yu, Guangzhou (CN)

Correspondence Address: Andrew O. Scheinman, Ph.D.;ROSENBAUM & ASSOCIATES, P.C., Suite #380, 650 Dundee Road, Northbrook, IL, 60062, US

(21) Appl. No.: 11/005,207

(22) Filed: Dec. 6, 2004

(51) Int. Cl. A01K 67/027

(52) U.S. Cl. 800/14

(57) ABSTRACT

The present invention is directed towards the use of the masked palm civet Paguma larvata (“civet”) as an animal model system for SARS, and is based on the novel demonstration of the present invention that civets may be infected with exogenous coronavirus, and that such infection produces SARS-like symptoms in these infected animals. The present invention is directed to a civet model system for the study of the infection, replication, and clinical effects of exogenously introduced human SARS-CoV coronavirus strains, civet SARS-CoV-like coronavirus strains, or variants or derivatives thereof, and to the development of vaccines (or other methods of prevention) or treatment of infection or transmission to other civets or humans of these human SARS-CoV coronavirus strains, civet SARS-CoV-like coronavirus strains, or variants or derivatives thereof.

### BACKGROUND OF THE INVENTION

Severe Acute Respiratory Syndrome (“SARS”) is a human respiratory disease of recent origin, widespread infectivity, recurring incidence, and significant mortality. Specifically, SARS was first observed in China in November, 2002 and, during this 2002-2003 outbreak, spread to more than 30 countries, leading to 8096 confirmed SARS cases and 774 confirmed SARS-related deaths. SARS has since been observed in a second outbreak between December, 2003, and January, 2004, demonstrating that the disease is recurrent, and continues to be of serious impact to worldwide human health.

Although the etiological agent responsible for SARS has recently been identified, extensively characterized, and ultimately classified as a new member of the coronaviral family of viruses, even with this knowledge there has, to date, been little progress in developing methods for treating or preventing this devastating disease. Specifically, studies have shown the source of SARS to be a new viral member of the coronavirus family, the human SARS coronavirus, i.e., the “SARS-CoV” coronavirus (synonymously, “SARS-CoV”). See, e.g., Rota et al., Science 300:1394 (2003), and Marra et al., Science 300:1399 (2003). However, despite this identification of the SARS-CoV coronavirus as the causative agent for SARS, and the subsequent identification of various components of this virus as potential targets for drugs development based on an understanding of the structure and components of this coronavirus, there has been little progress in the development of either drugs or vaccines for this disease.

One hurdle of particular significance for such therapeutic and vaccine development is the lack of a suitable animal model for studying the disease. Specifically, in order to understand the progression of SARS in humans, or to determine the efficacy of a SARS vaccine or drug treatment regimen, it is critical to have one or more suitable non-human animal model systems available for such studies. Although a number of such model systems have been considered, e.g., monkeys including cynomolgus macaques and African green monkeys, ferrets, cats, mice, and pigs, each of these animal model systems have disadvantages, including applicability to human SARS infections, cost, etc.

In this regard, a particularly useful animal model system for SARS might be based on the use of the masked palm civet, Paguma larvata (“civet”), in light of this animal's likely role as the source of the precursor of the human SARS-CoV coronavirus. Specifically, the early cases of SARS in both the 2002-2003 and 2003-2004 outbreaks were associated with patient exposure to these exotic food animals, suggesting that they are the vectors for transmission to humans of the SARS-CoV coronavirus, or a close relative of the SARS-CoV coronavirus. Moreover, it has been shown that civets indeed harbor a SARS-CoV-like coronavirus (the “civet SARS-CoV-like” coronavirus, “SARS-CoV-like” coronavirus, etc.) that is highly related to the human SARS-CoV coronavirus (99.8% RNA sequence homology), further suggesting the origin of the latter human form of the coronavirus from transmission of the former civet form. See, e.g., Guan et al., Science 302:276 (2003).

In light of this data implicating civets as the reservoir for transmission of the SARS-CoV virus to humans, a model system based on civets would allow for the study of a variety of unknown or poorly characterized aspects of SARS. For example, such a civet-based model system would allow for the study of the evolution of the civet SARS-CoV-like coronavirus in these animals, thereby allowing for the development of drugs either to prevent the species jump of this virus to humans, or to prevent the disease in civets by, e.g., vaccines, thereby minimizing risk in humans.

Just as significantly, such a civet-based model system might be used to study the behavior of “early-stage” human SARS-CoV coronaviral isolates, i.e., coronaviral isolates from patients from the earliest stages of the 2002-2003 or 2003-2004 epidemics which are highly similar to the civet SARS-CoV-like coronavirus, thereby allowing for the development of treatments or vaccines based on the properties of these early-stage isolates. Such a civet-based animal model system for SARS could theoretically also be applied to the study of “middle-stage” and “late-stage” human SARS-CoV coronaviral strains, i.e., strains isolated from patients infected later in either the 2002-2003 or 2003-2004 epidemics. Information on these strains would be particularly valuable because these strains are thought to be more adapted to reproduction and infection of the human host than are the early-stage coronaviral strains, i.e., better adapted than the early-stage civet-derived strains which are newly present in human hosts.

Despite these advantages for the use of civets as a model system for the study of SARS, to date there has been no demonstration of the workability of such a system, i.e., no demonstration that any exogenously introduced civet SARS-CoV-like coronavirus or early-, middle-, or late-stage strain of the human SARS-CoV coronavirus will infect civets. While the fact that the civet SARS-CoV-like coronavirus clearly does infect civets in the wild implicates its ability to infect laboratory animals, this observation does not, of itself, provide a sufficient basis for concluding the inevitability of infection in laboratory animals, especially given the unknown natural path of infection of civets in the wild. For the early-, middle-, and late-stage human strains the situation is even more uncertain, for there are at present no data indicating that such strains infect civets and, given their increasingly great divergences from the civet SARS-CoV-like strains, good reason to argue that these human strains, by adapting to the human host, have in fact lost all ability to reproduce in civets.

In light of the lack of a suitable animal model system for SARS and the benefits of a civet model system as described above, there is thus a strong need for the development of a civet-based model system for SARS. Specifically, there is a need to determine whether any civet SARS-CoV-like coronaviral strains or early-, middle-, and late-stage human SARS-CoV coronaviral strains are capable of infecting civets and, if so, what symptoms occur in these animals post-infection.

### SUMMARY OF THE INVENTION

The present invention derives from the novel observation presented in Example 1 that both early- and middle-stage human-derived SARS-CoV coronaviral strains are capable of producing SARS-like symptoms in civets. Specifically, the present invention presents the novel demonstration that both the early-stage human-derived SARS-CoV coronaviral strain GZ01 (which has the genomic sequence of SEQ ID NO:1) and the middle-stage human-derived SARS-CoV coronaviral strain BJ01 (which has the genomic sequence of SEQ ID NO:2) produce a variety of SARS-like symptoms in civets that have been infected with these coronaviral strains.

Thus one aspect of the present invention is directed to a civet model system for the study of the infection, replication, and clinical effects of exogenously introduced human SARS-CoV coronavirus strains, or variants or derivatives thereof, in civets, including early-, middle-, and late-stage human SARS-CoV coronavirus strains, and to the development of vaccines (or other methods of prevention) or treatment of infection or transmission to other civets or humans of these human SARS-CoV coronavirus strains, or variants or derivatives thereof.

In another aspect, the present invention is directed to a civet model system for the study of the infection, replication, and clinical effects of exogenously introduced civet SARS-CoV-like coronavirus strains, or variants or derivatives thereof, in civets, and to the development of vaccines (or other methods of prevention) or treatment of infection or transmission to other civets or humans of these civet SARS-CoV-like coronavirus strains, or variants or derivatives thereof.

### BRIEF DESCRIPTION OF THE DRAWINGS

The foregoing summary, as well as the following detailed description of preferred embodiments of the present invention, will be better understood when read in conjunction with the appended drawings. For the purpose of illustrating the present invention, there are shown in the drawings embodiments which are presently preferred. It should be understood, however, that the present invention is not limited to the precise arrangements and instrumentalities shown.

FIGS. 1A-B depict clinical changes in civets after inoculation with SARS coronavirus. (A) Daily average temperature of animals in groups A and B plotted together with the daily temperature of the control animal. Febrile episodes commenced around 3 days post-infection (“d.p.i.”), and temperatures remained elevated for up to 7 days in infected civets. (B) White blood cell (WBC) counts measured on day 0 and on days 3, 8 and 13 p.i. for the control animal and animals in groups A and B. For animals in groups A and B, the average counts are used in the plot. Leucopenia was observed with white blood cell counts reached minimum at approximately 3 d.p.i., and recovered to about normal level from 13 d.p.i.

FIGS. 2A-C present pathological changes in civets after inoculation with SARS-CoV. Lung tissues were taken on 13 d.p.i. from animal No. 5 of group A (A) and No. 7 of group B (B). Alveolar septa enlargement with macrophages and lymphocytes infiltration was evident in both animals. Lung tissue of the control animal (C) showed no abnormal changes. H. E. stain ×20.

FIG. 3 presents a table showing the detection of the SARS-CoV coronavirus in throat swab (T), anal swab (A), and blood (B) as determined by virus isolation or by RT-PCR. In this figure, and in FIG. 4, virus isolation/RT-PCR results are depicted, e.g., a table entry of “+/+” indicates that, for that entry (i.e., animal sampled at a particular d.p.i.), virus was detected by both viral isolation and RT-PCR, whereas a table entry of “−/+” indicates that, for that entry, no virus was isolated but, independently, RT-PCR detection indicated the presence of the viral sequence. In this figure, and in FIG. 4, “ND” denotes “not determined.”

FIG. 4 presents a table showing the detection of neutralizing antibodies in serum, and SARS-CoV in postmortem tissues. In determining neutralizing antibodies, all animals were verified prior to inoculation as lacking neutralizing antibodies to SARS-CoV in their pre-bleed sera. Virus isolation/RT-PCR terminology is as indicated for FIG. 3.

### DETAILED DESCRIPTION OF THE INVENTION

The present invention is directed towards the use of the masked palm civet Paguma larvata (“civet”) as an animal model system for SARS, and is based on the novel demonstration of the present invention that civets may be infected with exogenous coronavirus, and that such infection produces SARS-like symptoms in these infected animals.

Specifically, the present invention derives from the novel observation presented in Example 1 that both early- and middle-stage human-derived SARS-CoV coronaviral strains are capable of producing SARS-like symptoms in civets. Thus Example 1 demonstrates that both the early-stage human-derived SARS-CoV coronaviral strain GZ01 (GenBank accession number AY278489 (see the website ncbi.nlm.nih.gov/entrez/); this coronaviral strain has the genomic sequence of SEQ ID NO:1) and the middle-stage human-derived SARS-CoV coronaviral strain BJ01 (GenBank accession number AY278488 (see the website ncbi.nlm.nih.gov/entrez/); this coronaviral strain has the genomic sequence of SEQ ID NO:2) produce a variety of SARS-like symptoms in civets that have been infected with these coronaviral strains.

Thus, in one aspect, the present invention presents the novel observation that civets may be infected with human SARS-CoV coronavirus strains, and more specifically with both early-stage and middle-stage strains of the human SARS-CoV coronavirus. This observation is novel in light of the divergence of such human SARS-CoV coronavirus strains from coronavirus strains found in civets, i.e., from civet SARS-CoV-like coronavirus strains. Since such divergence is presumed to be a result of adaptation to the human host, it is therefore particularly unexpected that such human-adapted SARS-CoV strains are capable of infection of civets.

The present invention also demonstrates not only that such infection of civets by human SARS-CoV strains occurs, but further that such infection leads to SARS-like symptoms in these infected animals. See Example 1, below. This observation suggests that late-phase strains of human SARS-CoV may also infect civets, and serves as the basis for one broad embodiment of the invention directed to the study of civets infected with early-, middle-, and late-stage human SARS-CoV coronavirus strains, or variants or derivatives thereof, singly, or in combination. See, e.g., Examples 1-3 and 5.

A second broad embodiment of the present invention is also based on this novel observation that civets are capable of being infected with exogenous coronavirus, and is specifically directed to the study of civets infected with one or more civet SARS-CoV-like coronavirus strains, or variants or derivatives thereof. See, e.g., Examples 4-5.

Thus in one aspect, the present invention is directed to a civet model system for the study of the infection, replication, and clinical effects of exogenously introduced human SARS-CoV coronavirus strains, or variants or derivatives thereof, in civets, including early-, middle-, and late-stage human SARS-CoV coronavirus strains, and to the development of vaccines (or other methods of prevention) or treatment of infection or transmission to other civets or humans of these human SARS-CoV coronavirus strains, or variants or derivatives thereof. In another aspect, the present invention is directed to a civet model system for the study of the infection, replication, and clinical effects of exogenously introduced civet SARS-CoV-like coronavirus strains, or variants or derivatives thereof, in civets, and to the development of vaccines (or other methods of prevention) or treatment of infection or transmission to other civets or humans of these civet SARS-CoV-like coronavirus strains, or variants or derivatives thereof.

After a summary of the sequence identifiers and animals used in the present invention, each of these aspects of the present invention will be discussed.

Sequence Identifiers

In the present invention: SEQ ID NO:1 refers to the genomic sequence of the early-stage human-derived SARS-CoV coronavirus GZ01; SEQ ID NO:2 refers to the genomic sequence of the middle-stage human-derived SARS-CoV coronavirus BJ01; and, SEQ ID NOs:3-6 refer, respectively, to the VNUP, VNLOW, N355UP, and N355LOW PCR primers of Example 1.

Civets

As discussed above, the present invention is directed to the use of “civets.” As contemplated herein, this term refers to masked palm civets, which range from the Himalayan mountains to Indonesia, and which include at least four presently identified subspecies: P. larvata wroughtoni; P. larvata grayi; P. larvata neglecta; and P. larvata tytlerii. Also included in this term are other animals classified within this same species. Particularly preferred are those civets which are primarily found in China, i.e., P. larvata neglecta.

Infection of Civets

One aspect of the present invention is directed to the infection of civets with exogenous coronavirus, including exogenous human-derived coronavirus strains, and exogenous civet-derived coronavirus strains, as well as to civets that have been so infected.

As used herein, “infected” civets refers to civets which have been purposefully infected with one or more civet SARS-CoV-like coronavirus strain, one or more human SARS-CoV coronavirus strain (including strains corresponding to any of the various stages of infection in humans, e.g., early-, middle-, or late-phase strains, or any combination thereof), one or more variant or derivative of a civet SARS-CoV-like coronavirus strain or human SARS-CoV coronavirus strain, or some combination thereof. Synonymous terms used to describe such purposeful infection of civets include, e.g., “artificially infected,” “exogenously infected,” “infected with exogenous coronavirus,” “infected with exogenously introduced coronavirus,” etc. This terminology contemplates any method of purposeful infection, including, e.g., intratracheal, intranasal, or subcutaneous inoculation, as well as other routes of entry as might occur in the wild, e.g., infection via the respiratory tract, alimentary tract, skin bruises, etc., or any other route or method of infection as would be known to one of ordinary skill in the art. This terminology includes both “direct” infection of a civet by any of the routes described above, and “indirect” infection of a civet by purposeful exposure of that animal to a civet harboring a civet SARS-CoV-like coronavirus strain or human SARS-CoV coronavirus strain, or variants or derivatives thereof.

In producing an infection in civets to obtain an “infected” animal of the present invention, it will be necessary to optimize the dosage and administration regime for introducing the coronviral strain or strains of interest. Such optimization requires the use of different dosages of coronavirus, different administration regimes such as route of administration, number of dosages of administration, time between dosages, etc.

Such optimization of infection will also require one or more methods for assaying the “SARS-like” nature of the infection itself, i.e., for measuring: any of the clinical symptoms characteristic of a human SARS infection or of a corresponding infection in civets; measuring presence of virus in the animal's body by direct detection (e.g., PCR, RT-PCR, etc.) or via neutralizing antibodies; measuring tissue abnormalties/damage, etc. Thus “SARS-like” refers to the extent to which the symptoms, viral load, etc., observed in the infected civet resembles the symptoms, viral load, etc., that is expected as characteristic of SARS in this animal model. The particular characteristic in question will depend upon the criterion selected; the evaluation of whether an animal exhibits such “SARS-like” symptoms will be readily made by one of ordinary skill in the art.

Thus assays contemplated in the present invention include, but are not limited to: measuring clinical symptoms in the animal such as febrile episodes, lethargy, leucopenia, diarrhea, etc.; measuring coronaviral load in the animal via, e.g., pharyngeal and rectal swabs and blood samples; measuring the presence of neutralizing antibodies in serum; and, post-mortem analyses in, e.g., brain, lung, spleen, lymph nodes, kidney, and liver for both coronaviral presence and for indications of abnormalities/damage resulting from infection.

Examples of optimization as well as assays for measuring infection are provided in, e.g., Examples 1-3 below.

Coronaviral Strains

The present invention contemplates a variety of exogenous coronaviral strains for the infection of civets, including exogenous human-derived coronavirus strains, and exogenous civet-derived coronavirus strains.

“Exogenous coronavirus,” as used herein, refers to the strain or strains of coronavirus used to infect the civets of the present invention, and indicates a coronavirus that is exogenously introduced, i.e., introduced by “infection” as described above. Exogenous coronaviruses include, but are not limited to, “human-derived” SARS-CoV strains (including any of the various strains of human SARS-CoV isolated from, e.g., the 2002-2003 and/or 2003-2004 SARS epidemics), and “civet-derived” SARS-CoV-like strains. While such human- and civet-derived coronavirus strains are preferred in the present invention, other coronavirus strains are also contemplated, particularly those relatively closely related to civet SARS-CoV-like strains or human SARS-CoV strains. For examples of such related coronavirus strains see, e.g., the various coronaviruses presented in the phylogenetic analyses of Rota et al., Science 300:1394 (2003), or Marra et al., Science 300:1399 (2003).

Thus the present invention specifically contemplates the use of “human-derived” exogenous coronavirus strains, including human-derived strains classified as early-, middle-, or late-stage human SARS-CoV strains, i.e., strains which occurred successively later in the worldwide 2002-2003 SARS epidemic, and which, not coincidentally, correlate with increased adaptation to humans and therefore less similarity to the presumptive precursor civet SARS-CoV-like coronavirus. See Chinese SARS Molecular Epidemiology Consortium, Science 303:1666 (2004). Such strains may be defined by their occurrence during this epidemic; alternatively, they may be classified by characteristic nucleotide patterns in their roughly 30 kb RNA genome. For example, strains with a genome having the nucleotide pentet G₁₇₅₆₄A₂₁₇₂₁C₂₂₂₂₂G₂₃₈₂₃C₂₇₈₂₇ (i.e., G at nucleotide position 17564; A at nucleotide position 21721; C at nucleotide position 22222; G at nucleotide position 23823; and, C at nucleotide position 27827) relative to the human SARS-CoV early-stage coronavirus strain GZ02 (GenBank Accession No. AY390556, available at the website ncbi.nlm.nih.gov/entrez) may be classified as early-stage human SARS-CoV sequences; strains with a genome having the nucleotide pentet G₁₇₅₆₄A₂₁₇₂₁C₂₂₂₂₂T₂₃₈₂₃C₂₇₈₂₇ relative to GZ02 may be classified as middle-stage human SARS-CoV sequences; and, strains with a genome having the nucleotide pentet T₁₇₅₆₄G₂₁₇₂₁T₂₂₂₂₂T₂₃₈₂₃T₂₇₈₂₇ relative to GZ02 may be classified as late-stage human SARS-CoV sequences. See Chinese SARS Molecular Epidemiology Consortium, Science 303:1666 (2004).

Alternatively, human-derived early-, middle-, and late-stage SARS-CoV strains may be defined by other characteristics, or combinations of characteristics, for example by insertions or deletions in the nucleotide sequence of the RNA genome for such strains. For example, a 29 nucleotide (“nt”) sequence that is present in the genomes of early-stage human SARS-CoV strains and in civet SARS-CoV-like strains is deleted from the genomes of most middle- and late-stage human SARS-CoV strains. Thus middle- or late-stage human SARS-CoV strains may be determined by the absence of this nucleotide sequence. See Chinese SARS Molecular Epidemiology Consortium, Science 303:1666 (2004).

As contemplated herein, early-stage human SARS-CoV strains include, but are not limited to, GZ02, GZ01 (synonymously, “GD01”), HGZ8L1-A, HSZ-Cc, HSZ-A, HSZ-Bb, HSZ-Cb, HSZ-Bc, GZ50, GZ-A, JMD, HGZ8L1-B, ZS-A, ZS-B, and ZS-C; middle-stage human SARS-CoV strains include, but are not limited to, BJ04, BJ03, BJ02, BJ01, CUHK-W1, HZS2-D, HZS2-E, HZS2-C, HGZ8L2, HZS2-Bb, HSZ2-A, HZS2-Fc, and HZS2-Fb; and, late-stage human SARS-CoV strains include, but are not limited to, TWC, Sin2679, ZJ01, HSR, TW1, HKU-39849, GZ-D, Urbani, Sin2748, Sin2677, Sin2500, Frankfurt, Sin2774, CUHK-Su10, CUHK-LC1, CUHK-AG01, CUHK-AG02, CUHK-AG03, TWH, TC1, TWY, TWS, TWK, TWJ, TC3, TC2, GZ-B, GZ-C, TOR2, CUHK-LC2, CUHK-LC3, CUHK-LC4, and CUHK-LC5. See, e.g., Chinese SARS Molecular Epidemiology Consortium, Science 303:1666 (2004). Thus the two coronaviral strains used in Example 1 below, GZ01 (which has the genomic sequence of SEQ ID NO:1) and BJ01 (which has the genomic sequence of SEQ ID NO:2), are, respectively, early-stage and middle-stage human SARS-CoV coronaviral strains.

The present invention also contemplates the use of “civet-derived” SARS-CoV-like strains. Such strains include, e.g., strains isolated from civets during the time frame of the 2002-2003 worldwide SARS epidemic, and strains isolated from civets during the time frame of the 2003-2004 SARS epidemic.

In addition to the sources of “human-derived” and “civet-derived” coronavirus strains discussed above, the present invention also contemplates other sources for such materials, particularly stored body tissue, fluids, etc., of humans or civets. Thus the term “human-derived” encompasses coronavirus strains obtained from fixed or frozen human tissue, preserved fluids, etc. Similarly, the term “civet-derived” encompasses coronavirus strains obtained from fixed or frozen civet tissue, fluids, etc.

Coronaviral Variants

The present invention also contemplates the infection of civets with coronaviral variants, including, e.g., variants of the human-derived and civet-derived coronaviral strains described above.

As used herein, “variant” refers to coronaviral strains which have a genomic RNA sequence that is varied from that of one or more previously characterized coronaviral reference strains. Such variants are particularly contemplated as including coronaviral strains where certain nucleotide positions, insertions, deletions, etc., are fixed (unvaried), while others are allowed to vary, with the total variation from the reference strain or strains small enough that the variant strains have genomes “substantially identical” (synonymously, “substantially similar”) to the reference genome(s). Such variants may be additionally required to have partial or complete functionality with regard to one or more of the functionalities exhibited by a complete coronavirus, or some component thereof, as described below.

Thus variants of the human-derived SARS-CoV coronaviral strains of the present invention or of the civet-derived SARS-CoV-like coronaviral strains of the present invention may include those coronaviral strains with genomes which preserve the hallmark features of a human early-, middle-, or late-stage coronaviral strain, or the features of a civet SARS-CoV-like coronaviral strain, while having some degree of variation over the remaining positions in some or all of the genome such that the genome or some part thereof is substantially identical to that of a reference coronaviral genome. For example, variants of early-stage human SARS-CoV coronaviral strains include sequences that are required to have the fixed nucleotide pentet G₁₇₅₆₄A₂₁₇₂₁ C₂₂₂₂₂G₂₃₈₂₃C₂₇₈₂₇ that is characteristic of early-stage human SARS-CoV sequences while having varied nucleotides elsewhere in the genome such that the sequence is substantially similar to a reference early-stage human SARS-CoV coronavirus genomic sequence, e.g., the GZ02 sequence. Similarly, variants of middle-stage human SARS-CoV coronaviral strains include sequences that are required to have the fixed nucleotide pentet G₁₇₅₆₄A₂₁₇₂₁C₂₂₂₂₂T₂₃₈₂₃C₂₇₈₂₇ that is characteristic of middle-stage human SARS-CoV sequences while having varied nucleotides elsewhere in the genome such that the sequence is substantially similar to a reference middle-stage human SARS-CoV coronavirus genomic sequence, while variants of late-stage human SARS-CoV coronaviral strains include sequences that are required to have the fixed nucleotide pentet T₁₇₅₆₄G₂₁₇₂₁T₂₂₂₂₂T₂₃₈₂₃T₂₇₈₂₇ that is characteristic of late-stage human SARS-CoV sequences while having varied nucleotides elsewhere in the genome such that the sequence is substantially similar to a reference late-stage human SARS-CoV coronavirus genomic sequence.

Variants of the coronaviral strains of the invention may also include other fixed features, such as, e.g., the presence or absence of the 29 nt region that characterizes, respectively, early-stage versus middle-stage/late-stage human SARS-CoV coronaviral strains. Also contemplated are other such fixed features characteristic of various strains of coronavirus, as well as combinations of such features.

As discussed above, the term “variants” as contemplated herein refers to coronaviral genomic sequences that are allowed to vary, particularly at non-fixed positions. Although the invention contemplates such variation as allowing for the equal selection at any particular nucleotide position of all four allowable nucleotides, particularly contemplated are variations that preserve the nucleotide usage characteristic of the coronavirus (see Yap et al., BMC Informatics 4:43 (2003)), as well as variations that preserve the protein-encoding properties of the nucleotide sequence. Thus variations in sequence positions that occur in protein-encoding regions of the genome are preferably made in order to preserve the identity of the amino acid in the corresponding region of the encoded polypeptide, i.e., a C that occurs in the coronaviral sequence as the first nucleotide in the arginine-encoding triplet CGA is preferentially varied to an A in order to ensure that the resulting triplet AGA still encodes arginine in the corresponding polypeptide. The choice of such variations in order to preserve the amino acid-encoding properties of the genomic sequence is based on the triplet genetic code, and is well-known to one of ordinary skill in the art.

In addition to the preservation of protein sequence discussed above, the present invention contemplates other limitations on variation so as to preserve other functionalities of the coronaviral sequence. Thus, for example, variations in the genomic sequence in protein-encoding regions may be made that, while changing one or more amino acids of the encoded protein, do not result in loss of function or functions of that protein. Such preservation of protein functionality may be accomplished in a variety of ways. For example, amino acid substitutions may be conservative substitutions, i.e., substitutions of amino acids with other amino acids with similar properties. The skilled artisan will understand that such conservative substitutions will be more important for amino acids whose chemical properties are important for protein function than for those amino acid positions where the particular properties of the amino acid at that position are not as critical to protein function.

The present invention also contemplates other functionalities of the coronavirus as being important for preservation, i.e., for preservation in a variant coronavirus. For example, during the life-cycle of the SARS-CoV or SARS-CoV-like coronavirus a polyprotein is produced that is cleaved by coronaviral-encoded proteinases to yield the individual protein components of, e.g., the viral replication complex. See, e.g., Stadler et al., Nature Rev. Microbiol. 1:213 (2003). Thus in one embodiment, coronaviral variants have the additional requirement of preserving the nucleotide positions necessary to produce a corresponding polyprotein in which the proteinase cleavage sites are preserved. Other examples of functionalities that may be preserved in the coronaviral variants of the present invention are given in, e.g., Rota et al., Science 300:1394 (2003), Marra et al., Science 300:1399 (2003), and Stadler et al., Nature Rev. Microbiol. 1:213 (2003).

When a particular protein function or functions is to be preserved in a variant coronavirus of the invention, a variety of assays may be used either to demonstrate such preservation, or, when used as a selection assay, to generate a variant coronavirus in which that function or functions are maintained. Thus for example, preservation of the proteinase function described above may be assayed in vitro, e.g., by determining whether appropriate coronaviral protein fractions exhibit such proteinase activity, or in vivo, by assays viral replication. Such assays may also be used in combination with mutagenesis or other variant-generating techniques to create variants preserving the desired functionality or functionalities without undue effort.

In addition to the factors influencing the choice of variants discussed above, the present invention also contemplates that variants will have genomes substantially identical to a chosen coronaviral reference genome. For example, variants of the GZ01 early-stage and BJ01 middle-stage SARS-CoV coronviral strains of Example 1 are contemplated to include coronaviruses that have genomic sequences substantially identical to either the GZ01 or BJ01 genomic sequences. Such “substantial identity” refers to a high % sequence identity between the variant sequence and the reference sequence. Thus for example the present invention contemplates situations in which the % identity between the non-fixed positions of the variant sequence and the reference sequence is at least 90%, 91%, 92%, 93%, 94%, 95%, 96%, 97%, 98%, 99%, 99.1%, 99.2%, 99.3%, 99.4%, 99.5%, 99.6%, 99.7%, 99.8%, or 99.9%. This % identity may be judged by an alignment over the entire length of the SARS-CoV or SARS-CoV-like RNA sequence (i.e., over the approximately 29,000 bases of the RNA sequence), or it may be determined over a shorter length of the sequence, for example, 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, etc. (i.e., continuing by increments of 10 nucleotides up to the maximum length of the RNA). This % identity may be calculated by one of the algorithms described below; alternatively, it may be calculated as the number of different nucleotides per 100 nucleotides, such that a % identity of 99.9% would refer to no more than 1 nucleotide difference per 1000 nucleotides.

With regard to “% identity,” the following terms are used to describe the sequence relationships between two or more nucleic acids, polynucleotides, or polypeptides: “reference sequence”; “comparison window”; “sequence identity”; “percentage of sequence identity”; and, “substantial identity.” Note that this discussion is explicitly intended to encompass both polynucleotide and polypeptide sequences.

Thus as used herein, “reference sequence” is a defined sequence used as a basis for sequence comparison. A reference sequence may be a subset or the entirety of a specified sequence; for example, as a segment of a full-length cDNA or gene sequence, or the complete cDNA or gene sequence. Thus reference sequences of the present invention include SARS-CoV and SARS-CoV-like sequences, as well as subsets of these sequences, such as fragments or variants. By “fragment” is intended a portion of a nucleotide or amino acid sequence of the present invention.

As used herein, “comparison window” makes reference to a contiguous and specified segment of a polynucleotide sequence, wherein the polynucleotide sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the polynucleotide sequence a gap penalty is typically introduced and is subtracted from the number of matches. The present invention contemplates that analogous considerations will apply to polypeptide sequences.

Methods of alignment of sequences for comparison are well known in the art. Thus, the determination of percent sequence identity between any two sequences can be accomplished using a mathematical algorithm. Preferred, non-limiting examples of such mathematical algorithms are the algorithm of Myers and Miller (1988) CABIOS 4:11-17; the local homology algorithm of Smith et al., Adv. Appl. Math. 2:482 (1981); the homology alignment algorithm of Needleman and Wunsch (1970) J. Mol. Biol. 48:443-453; the search-for-similarity-method of Pearson and Lipman (1988) Proc. Natl. Acad. Sci. 85:2444-2448; and, the algorithm of Karlin and Altschul (1990) Proc. Natl. Acad. Sci. USA 872264, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wis., USA). Alignments using these programs can be performed using the default parameters. The CLUSTAL program is well described by Higgins et al. (1988) Gene 73:237-244 (1988); Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See www.ncbi.nlm.nih.gov. Alignment may also be performed manually by inspection.

For purposes of the present invention, comparison of nucleotide or protein sequences for determination of percent sequence identity to the polynucleotide or polypeptide sequences disclosed herein is preferably made using the Clustal W program (Version 1.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.

As used herein, “sequence identity” or “identity” in the context of two nucleic acid or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, Calif.).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

The term “substantial identity” of polynucleotide sequences means that a polynucleotide comprises a sequence that has at least 70% sequence identity, preferably at least 80%, more preferably at least 90%, and most preferably at least 95%, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 60%, more preferably at least 70%, 80%, 90%, and most preferably at least 95%.

An additional indication that nucleotide sequences are substantially identical is if two molecules hybridize to each other under stringent conditions.

Thus in hybridization, all or part of a known nucleotide sequence is used as a probe that selectively hybridizes to other corresponding nucleotide sequences, e.g., a SARS-CoV or SARS-CoV-like coronaviral cDNA sequence. In general hybridization probes may be genomic DNA fragments, cDNA fragments, RNA fragments, or other oligonucleotides, as appropriate, and may be labeled with a detectable group such as ³²P, or any other detectable marker. Thus, for example, probes for hybridization can be made by labeling synthetic oligonucleotides based on the coronaviral sequences of the invention. Methods for preparation of probes for hybridization and PCR are generally known in the art and are disclosed in Sambrook et al. (1989) Molecular Cloning: A Laboratory Manual (2d ed., Cold Spring Harbor Laboratory Press, Plainview, N.Y.).

An important parameter in hybridizations is the specificity of hybridization between the template and probes. Thus to achieve specific hybridization under a variety of conditions, such probes include sequences that are unique to the desired region of the coronaviral sequence, and are preferably at least about 10 nucleotides in length, and most preferably at least about 20 nucleotides in length. In PCR reactions, such probes may be used to amplify corresponding coronaviral sequence regions of interest, or as a diagnostic assay to determine the presence of particular sequence regions or individual nucleotides in a coronaviral template nucleotide sequence.

Hybridizations may be carried out under different conditions of stringency, for example under stringent conditions. By “stringent conditions” or “stringent hybridization conditions” is intended conditions under which a probe will hybridize to its target sequence to a detectably greater degree than to other sequences (e.g., at least 2-fold over background). Stringent conditions are sequence-dependent and will be different in different circumstances. By controlling the stringency of the hybridization and/or washing conditions, target sequences that are 100% complementary to the probe can be identified (homologous probing). Alternatively, stringency conditions can be adjusted to allow some mismatching in sequences so that lower degrees of similarity are detected (heterologous probing). Generally, a probe is less than about 1000 nucleotides in length, preferably less than 500 nucleotides in length.

Typically, stringent conditions will be those in which the salt concentration is less than about 1.5 M Na ion, typically about 0.01 to 1.0 M Na ion concentration (or other salts) at pH 7.0 to 8.3 and the temperature is at least about 30° C. for short probes (e.g., 10 to 50 nucleotides) and at least about 60° C. for long probes (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulphate) at 37° C., and a wash in 1× to 2×SSC (20×SSC=3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5× to 1×SSC at 55 to 60° C. Exemplary high stringency conditions include hybridization in 50% formamide, 1 M NaCl, 1% SDS at 37° C., and a wash in 0.1×SSC at 60 to 65° C. Duration of hybridization is generally less than about 24 hours, usually about 4 to about 12 hours.

Specificity is typically the function of post-hybridization washes, with the critical factors being the ionic strength and temperature of the final wash solution. For DNA-DNA hybrids, the Tₘ can be approximated from the equation of Meinkoth and Wahl (Anal. Biochem. 138:267 (1984)): Tₘ=81.5° C.+16.6 (log M)+0.41 (% GC)-0.61 (% form)-500/L; where M is the molarity of monovalent cations, % GC is the percentage of guanosine and cytosine nucleotides in the DNA, % form is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization, and/or wash conditions can be adjusted to hybridize to sequences of the desired identity. For example, if sequences with about 90% identity are sought, the Tₘ can be decreased 10° C. Generally, stringent conditions are selected to be about 5° C. lower than the thermal melting point (Tₘ) for the specific sequence and its complement at a defined ionic strength and pH. However, severely stringent conditions can utilize a hybridization and/or wash at 1, 2, 3, or 4° C. lower than the thermal melting point (Tₘ); moderately stringent conditions can utilize a hybridization and/or wash at 6, 7, 8, 9, or 10° C. lower than the thermal melting point (Tₘ); and, low stringency conditions can utilize a hybridization and/or wash at 11, 12, 13, 14, 15, or 20° C. lower than the thermal melting point (Tₘ). Using the equation, hybridization and wash compositions, and desired Tₘ those of ordinary skill will understand that variations in the stringency of hybridization and/or wash solutions are inherently described. If the desired degree of mismatching results in a Tₘ of less than 45° C. (aqueous solution) or 32° C. (formamide solution), it is preferred to increase the SSC concentration so that a higher temperature can be used. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, N.Y.); and Ausubel et al., eds. (1995) Current Protocols in Molecular Biology, Chapter 2 (Greene Publishing and Wiley-Interscience, New York). See also Sambrook et al. (1989) Molecular Cloning: A Laboratory Manual (2d ed., Cold Spring Harbor Laboratory Press, Plainview, N.Y.).

Thus, as discussed above, an indication that nucleotide sequences are substantially identical is if two molecules hybridize to each other under stringent conditions. Generally, stringent conditions are selected to be about 5° C. lower than the thermal melting point (Tₘ) for the specific sequence at a defined ionic strength and pH. However, stringent conditions encompass temperatures in the range of about 1° C. to about 20° C. lower than the Tₘ, depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.

Coronaviral Derivatives

In addition to the coronaviral variants discussed above, the present invention also contemplates the use of coronaviral “derivatives.” Such derivatives are specifically contemplated to include derivatives of a coronaviral genome, e.g., regions of the genome encoding particular proteins or sets of proteins such as the polyprotein, structural regions, etc., where such regions are used in isolated form or within vectors for their replication, expression, etc. Such derivatives are also contemplated to include derivatives of the set of coronaviral proteins, e.g., one or more coronaviral proteins, or modified forms thereof, as would for example be used in vaccine development.

Thus one aspect of the present invention is drawn to derivatives of the coronaviral genome such as particular protein-encoding regions of the SARS-CoV genome, either in isolated form or contained within, e.g., an expression vector. For example, one aspect of the invention is directed to the portion of the human early-, middle-, or late-stage SARS-CoV coronaviral genome or of the civet SARS-CoV-like genome encoding the spike or “S” protein, i.e., the protein that is thought to mediate both biding to host cell receptors and membrane glycoprotein fusion upon binding. Thus for example coronaviral derivatives comprising this S-protein-encoding region within an expression vector have utility for studying the action in civets of either the human- or civet-derived S-protein polypeptides produced from these expression vectors. Such vectors also have utility in vaccine development, specifically for the production in healthy civets of an immune reaction to the expressed S-protein, thereby allowing for the possible protection of those animals against later challenge with various human-derived or civet-derived SARS-CoV or SARS-CoV-like strains. See, e.g., Bukreyev et al., Lancet 363:2122 (2004), for an example of such a system in African green monkeys.

In another aspect, the present invention is directed to coronaviral derivatives which comprise regions of the coronaviral genome encoding part, but not all, of a particular coronaviral protein. For example, it has been shown that the SARS-CoV S-protein is similar to previously characterized class I viral fusion proteins, suggesting that particular regions of importance to function in these class I viral fusion proteins may also be important for SARS-CoV S-protein function. Thus the present invention contemplates the use of coronaviral variants with one or more of these regions, for example one or more heptad repeat (“HR”) regions, which have been shown in vitro to inhibit SARS-CoV infection of African green monkey cells (“Vero” cells). See, e.g., Bosch et al., Proc. Natl. Acad. Sci. U.S.A. 101:8455. For example, the use of such coronaviral derivatives may be used in vectors to afford protection to civets against later infection with SARS-CoV coronaviral strains, SARS-CoV-like coronaviral strains, etc.

In addition to derivatives of the coronaviral genome, the present invention also contemplates derivatives of the set of coronaviral proteins, e.g., one or more coronaviral proteins, or modified forms thereof, as would for example be used in vaccine development. Thus S-protein may be directly used in isolated form in a civet, as well as produced by an expression vector comprising the S-protein encoding region of a coronaviral genome as discussed above. Similarly, fragments of one or more coronaviral proteins may be used directly in a civet, e.g., to provoke an immune reaction etc., rather than being produced in the animal from an expression vector comprising those protein-encoding regions of the coronaviral genome.

In light of the above discussions, it is clear that the present invention encompasses isolated or substantially purified nucleic acid or protein compositions. An “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. Preferably, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein having less than about 30%, 20%, 10%, 5%, (by dry weight) of contaminating protein. When the protein of the invention or biologically active portion thereof is recombinantly produced, preferably, culture medium represents less than about 30%, 20%, 10%, or 5% (by dry weight) of chemical precursors or non-protein of interest chemicals.

Also in light of the above discussions, it should be noted that the coronaviral “derivatives” of the present invention include derivatives of variant coronaviruses. Thus for example any variant coronaviral genome as defined above may also serve as the basis for a coronaviral genome derivative.

Cells and Coronaviruses Obtained from Infected Civets

The present invention contemplates the use of material obtained from infected civets, including, e.g., cells of these infected animals, and coronaviral strains derived from these infected animals.

Thus one aspect of the present invention is directed to cells obtained from infected civets, as would be used in, for example, in vitro assays for drug or vaccine development. Specifically, the use of cells obtained from infected civets is often preferable to the use of the infected animals themselves; thus one aspect of the invention is directed specifically to these cells obtained from infected animals using any of the techniques known to one of ordinary skill in the art of cell biology and/or tissue culture.

The present invention also contemplates the use of coronaviral strains obtained from infected civets, particularly variant coronaviral strains obtained during the course of infection of the animal. Thus it is known that RNA viruses such as coronaviruses have a high mutation rate, such that initial infection of an animal with a particular defined strain will, over the course of infection, frequently result in the production of a variety of variant strains within the animal. The analysis of such variation during infection can be expected to shed light on how the civet-derived or human-derived coronaviral strain chosen for initial infection evolves in the animal, thereby offering up, e.g., insights in how to treat or prevent the disease. Thus the present invention contemplates the isolation of these variant strains during the course of infection, e.g., by withdrawing blood from the animal daily and isolating and characterizing the coronaviral content of the blood for each of these time points.

Although cells and coronaviruses derived from infected civets are explicitly contemplated in the invention, the invention is not limited to these isolates from civets, but includes other isolates, e.g., isolated fluids such as blood, as well as urine, feces, etc

Drug Development

The human-derived SARS-CoV coronaviral-infected or civet-derived SARS-CoV-like coronaviral-infected civets of the present invention may be used in the development of drugs for protection against the onset of one or more SARS-like symptoms in these animals.

Thus one aspect of the present invention is directed to the development of such “coronaviral protection agents” for protecting civets infected with exogenous coronavirus from one or more of the SARS-like symptoms associated with the presence of such human-derived SARS-CoV coronavirus or civet-derived SARS-CoV-like coronavirus in these animals. “Coronaviral protection agent,” as contemplated herein, refers to any agent capable of protecting such infected civets, i.e., capable of reducing or eliminating one or more of the SARS-like symptoms seen in these animals. Thus for example “coronaviral protection agent” includes an agent capable of preventing replication of a human-derived SARS-CoV coronavirus or civet-derived SARS-CoV-like coronavirus strain or strains in an infected animal, an agent capable of reducing or eliminating the secondary effects of such viral reproduction in an infected animal, etc.

The skilled artisan will recognize that putative “coronaviral protection agents” include any agents known to have activity or potential activity against coronaviruses, or their effects, including, e.g., inhibitors of the proteinase that cleaves the coronaviral polyprotein, antibodies capable of inactivating the coronavirus, blocking the binding of the coronavirus mediated by the S-protein, etc.

For the present invention, “coronaviral protection agents” will be determined by assaying the onset of SARS-like symptoms in animals that have been treated with a putative coronaviral protection agent before, concurrent with, or after infection with the coronavirus strain or strains of interest. Thus for example animals may be treated with a putative coronaviral protection agent capable of blocking cleavage of the coronaviral polyprotein, after which the animals are infected with one or more coronaviral strains of interest, and assayed for the development of one or more SARS-like symptoms. In such an assay, a putatitve coronaviral protection agent that elicts an actual reduction in one or more SARS-like symptoms will be classified as an actual coronaviral protection agent.

The present invention contemplates the development of coronaviral protection agents using the whole animal assays described above, and also via the use of isolated civet cells, either from animals free of coronavirus or animals that have been infected with exogenous coronavirus. For example, civet cells in culture may be used as a model system instead of whole animals, i.e., cultured cells may be pretreated with putative coronaviral protection agent, followed by infection with exogenous coronavirus and screening for SARS-like symptoms. The skilled artisan will understand that in such in vitro assays, relevant symptoms will be a subset of those seen in whole animals, for example, coronaviral load, expression of particular coronaviral proteins, production of various cellular enzymes in response to infection with exogenous coronavirus, etc.

Vaccine Development

The human-derived SARS-CoV coronaviral-infected or civet-derived SARS-CoV-like coronaviral-infected civets of the present invention may be used in the development of vaccines for protection against the onset of one or more SARS-like symptoms in these animals. This application is related to the drug development aspect of the invention discussed above; therefore, it is to be understood that the discussion in that section of the present invention also applies herein (and vice-versa).

As for the drug development discussed above, the development of vaccines in the civet animal model system of the invention is based on the use of whole animals or on in vitro assays using cultured civet cells to determine the protective effects of various putative “coronaviral vaccine agents” against one or more human-derived SARS-CoV coronaviral strains or civet-derived SARS-CoV-like coronaviral-infected strains of the present invention. Thus putative coronaviral vaccine agents contemplated in the present invention include agents based on whole-coronaviruses or variants thereof, including live-attenuated and inactivated coronaviruses. The present invention also contemplates the use of various components of the coronavirus, e.g., the S protein. Also contemplated are vaccines based on antibodies against the coronavirus, or component or components thereof (see, e.g., ter Meulen et al., Lancet 363:2139 (2004)).

In the whole-coronavirus vaccines of the present invention, the coronavirus is mixed with the appropriate adjuvant, diluents, and carriers. Physiologically acceptable media that can be used include, but are not limited to, appropriate isoosmotic solutions and phosphate buffers. Vaccines based on components of the coronavirus, such as those based on the earliest stage S protein sequence, as described in the preceding section, are also contemplated herein. Thus for example a vector containing the nucleotide sequence encoding a human- or civet-derived coronaviral S protein under control of a suitable promoter and other gene regulatory sequences as would be known to the skilled artisan may be used to infect a civet; alternatively, isolated S protein may be introduced, etc.

The term “adjuvant” refers to a compound or mixture that enhances the immune response to an antigen. An adjuvant can serve as a tissue depot that slowly releases the antigen and also as a lymphoid system activator that non-specifically enhances the immune response (Hood et al., Immunology, Second Ed., 1984, Benjamin/Cummings: Menlo Park, Calif., p. 384). Often, a primary challenge with an antigen alone, in the absence of an adjuvant, will fail to elicit a humoral or cellular immune response. Adjuvants include, but are not limited to, complete Freund's adjuvant, incomplete Freund's adjuvant, saponin, mineral gels such as aluminum hydroxide, surface active substances such as lysolecithin, pluronic polyols, polyanions, peptides, oil or hydrocarbon emulsions, keyhole limpet hemocyanins, dinitrophenol, and potentially useful human adjuvants such as BCG (bacille Calmette-Guerin) and Corynebacterium parvum.

Selection of an adjuvant depends on the subject to be vaccinated. Preferably, a pharmaceutically acceptable adjuvant is used. For example, a vaccine for a human should avoid oil or hydrocarbon emulsion adjuvants, including complete and incomplete Freund's adjuvant. One example of an adjuvant suitable for use with humans is alum (alumina gel). In a specific embodiment, infra, the vaccine of the present invention is administered intramuscularly in alum. Alternatively, the vaccine of the present invention can be administered subcutaneously, intradermally, intraperitoneally, or via other acceptable vaccine administration routes.

A vaccine formulation may be administered to a subject per se or in the form of a pharmaceutical or therapeutic composition. Pharmaceutical compositions comprising the adjuvant of the invention and an antigen may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the antigens of the invention into preparations which can be used pharmaceutically. Proper formulation is dependent upon the route of administration chosen. For purposes of this application, “physiologically acceptable carrier” encompasses carriers that are acceptable for human or animal use without relatively harmful side effects (relative to the condition being treated), as well as diluents, excipients or auxiliaries that are likewise acceptable. Systemic formulations include those designed for administration by injection, e.g. subcutaneous, intradermal, intramuscular or intraperitoneal injection. For injection, the vaccine preparations may be formulated in aqueous solutions, preferably in physiologically compatible buffers such as Hanks's solution, Ringer's solution, phosphate buffered saline, or any other physiological saline buffer. The solution may contain formulatory agents such as suspending, stabilizing and/or dispersing agents. Alternatively, the proteins may be in powder form for constitution with a suitable vehicle, e.g., sterile pyrogen-free water, before use.

Determination of an effective amount of the vaccine formulation for administration is well within the capabilities of those skilled in the art. An effective dose can be estimated initially from in vitro assays. For example, a dose can be formulated in animal models to achieve an induction of an immune response using techniques that are well known in the art. One having ordinary skill in the art could readily optimize administration to all animal species based on results described herein. Dosage amount and interval may be adjusted individually. For example, when used as a vaccine, the vaccine formulations of the invention may be administered in about 1 to 3 doses for a 1-36 week period. Preferably, 1 or 2 doses are administered, at intervals of about 3 weeks to about 4 months, and booster vaccinations may be given periodically thereafter. Alternative protocols may be appropriate for individual animals. A suitable dose is an amount of the vaccine formulation that, when administered as described above, is capable of raising an immune response in an immunized animal sufficient to protect the animal from an infection for at least 4 to 12 months. In general, the amount of the antigen present in a dose ranges from about 1 pg to about 100 mg per kg of host, typically from about 10 pg to about 1 mg, and preferably from about 100 pg to about 1 pg. Suitable dose range will vary with the route of injection and the size of the patient, but will typically range from about 0.1 mL to about 5 mL.

### EXAMPLE 1

### Early- and Middle-Stage Human SARS-CoV Coronavirus Strains GZ01 and BJ01 are Able to Infect Civets and Produce SARS-Like Symptoms

As discussed above, although civets are known to harbor the civet SARS-CoV-like coronavirus, which is both a close relative to and the likely progenitor of the human SARS-CoV coronavirus, it has not been demonstrated that civets can be infected with this coronavirus strain, a necessary first step in using these animals to develop a model system for the study of SARS. Moreover, it has also not been determined whether the human SARS coronavirus SARS-CoV is able to infect civets and, if so, whether the middle- and late-stage strains of SARS-CoV, which are presumed to be more adapted to the human host, will be infective as compared to the early-stage strain of SARS-CoV.

This Example demonstrates for the first time that human SARS-CoV coronaviruses are in fact able to infect civets, and that infection by these SARS-CoV strains produces SARS-like symptoms in the infected animals. Moreover, this Example demonstrates that infectivity is not confined to early-stage human SARS-CoV coronaviruses, but also results when a middle-stage SARS-CoV coronavirus is used.

Specifically, human SARS-CoV coronaviral isolates GZ01 (which has the genomic sequence of SEQ ID NO:1) and BJ01 (which has the genomic sequence of SEQ ID NO:2) were used in this study. These isolates were originally obtained in Vero E6 cells at the Institute of Microbiology and Epidemiology, Academy of Military Medical Sciences, Beijing, and were propagated in Vero E6 cells for two additional passages to generate virus stocks with titers of 10⁶ 50% tissue culture infective doses (“TCID₅₀”) per ml. BJ01 contains the 29-nt deletion found in most middle- or late-stage human SARS-CoV isolates, whereas GZ01 resembles the viruses isolated from civets in that it does not have the 29-nt deletion.

To test the susceptibility of civets to SARS-CoV, eleven masked palm civets (Paguma larvata) were purchased from a farm in Hebei Province. All animals were approximately one year old, and none contained anti-SARS-CoV antibodies when tested by virus neutralization prior to the infection experiment. The animals were observed in the laboratory for approximately one month. No clinical signs were detection during the observation period and no SARS-CoV related RNA was detected in throat or anal swabs when analyzed by RT-PCR. Ten animals were each housed in separate biosafety isolators and divided into two groups (n=5 per group). Animals in group A and B were infected with 3 ml of virus solution containing 3×10⁶ TCID₅₀ of GZ01 and BJ01 isolates, respectively, with 2 ml given intratracheally and 1 ml intranasally. A control civet was mock infected in an identical fashion with 3 ml of Vero E6 cell culture supernatant. Animal experiments were conducted in accordance with animal ethics guidelines and approved protocols by the Harbin Veterinary Research Institute, Chinese Academy of Agricultural Sciences, and were carried out in an approved animal biosafety level 3 facility.

The clinical signs of the animals were checked daily. Throat swabs, anal swabs, and blood samples were taken on 0, 3, 8, 13, 18, 23, 28 and 33 days post-infection (“d.p.i”), and were subjected to virus isolation and RT-PCR analysis. Blood samples were also subjected to leukocyte counting. One animal from each group was sacrificed on 3, 13, 23, 34 and 35 d.p.i. On the day of euthanasia, lung, heart, spleen, lymph nodes, kidney and liver samples were taken from each animal and homogenized in PBS for virus isolation and RT-PCR analysis. Serum samples were also taken for virus neutralization analysis.

Clinical Symptoms

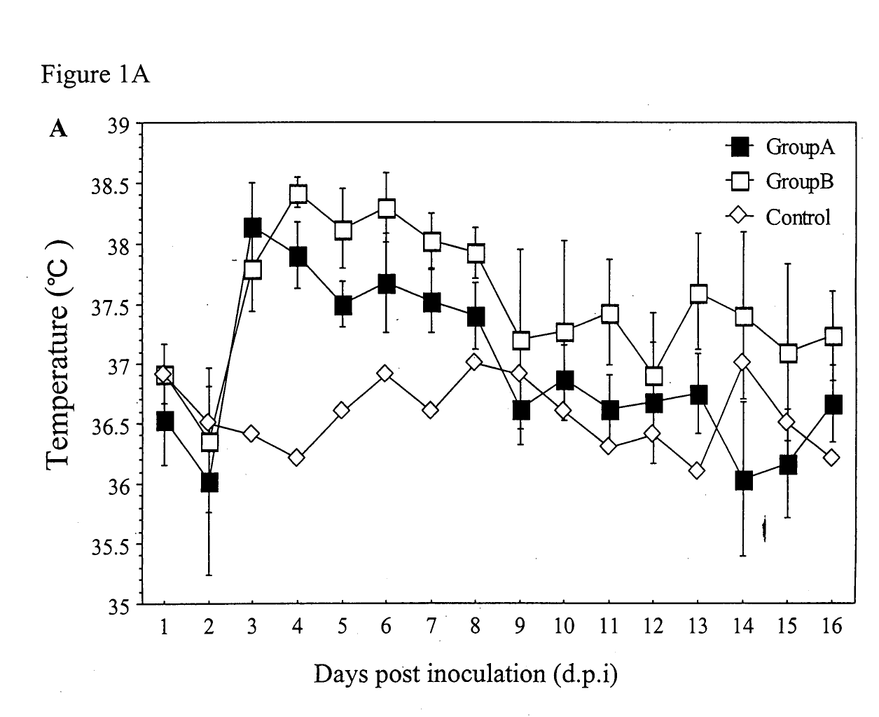

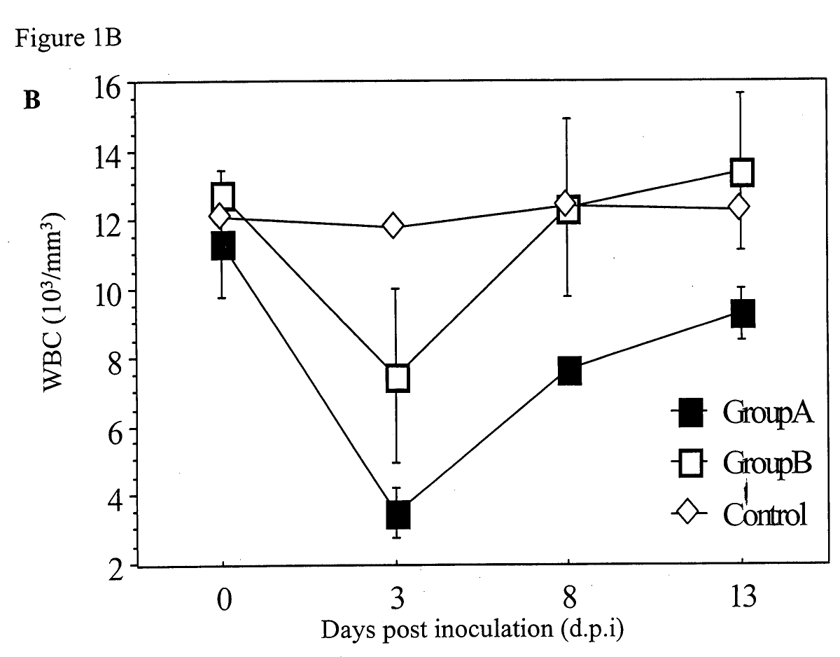

From 3 d.p.i., all animals became lethargic and less aggressive. Febrile episodes commenced around 3 d.p.i., and temperatures remained elevated for up to 7 days in infected civets (see FIG. 1A). Leucopenia was also observed with white blood cell counts reaching a minimum at approximately 3 d.p.i., and returning to normal from 13 d.p.i. onwards (see FIG. 1B). This trend was similar for animals in both group A and B. Two civets (No. 14 and 8) in group A and one in group B developed diarrhea between 3-14 d.p.i. Conjunctivitis was observed for one animal (No. 14) in group A and two (No. 7 and 11) in group B.

Histology

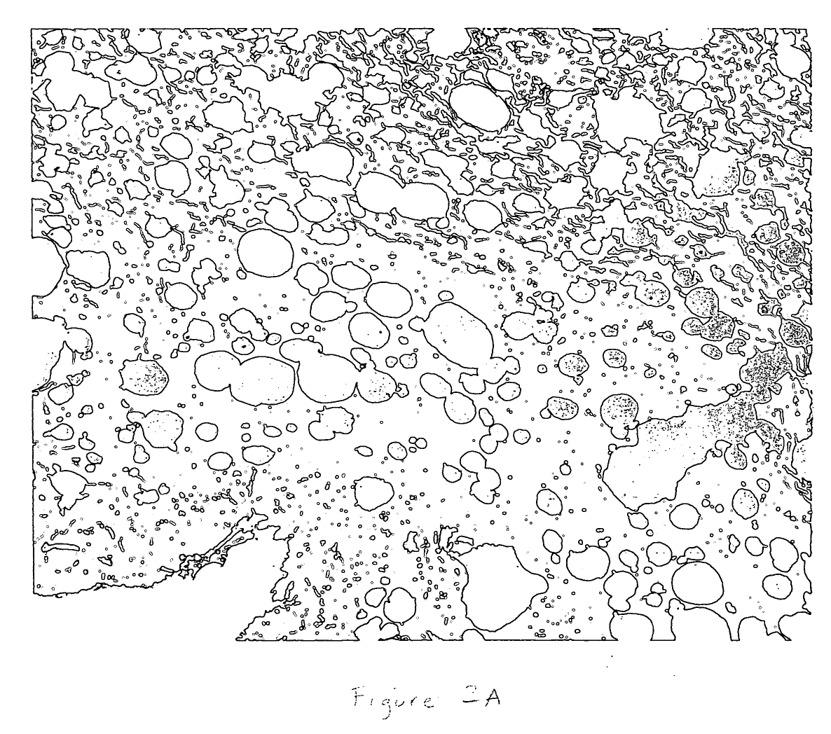

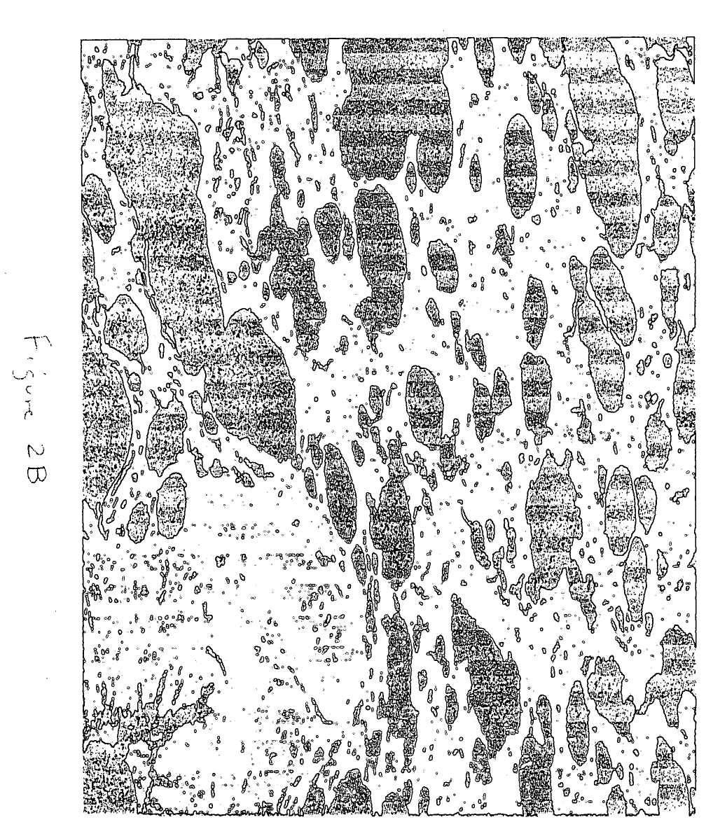

For histological examination, lung tissues were fixed in 10% neutral-buffered formalin, embedded in paraffin, and processed for routine histology. No gross pathological changes were found in necropsied animals. Histologically, interstitial pneumonia lesions were observed in both groups of animals on days 13-35 p.i. The lesions were similar to those described in the SARS-CoV-infected macaques, but the absence of syncytia (see FIGS. 2A and 2B) resembled the observation made in experimentally infected ferrets.

Virus Isolation

For virus isolation, collected samples were inoculated on Vero E6 cell monolayers in 96-well plates and passaged up to three times. For samples showing cytopathic effect (CPE), the presence of SARS-CoV was confirmed by electron microscopy and RT-PCR analysis. For RT-PCR analysis, viral RNA was isolated from swabs, serum, supernatants of homogenates and tissue culture using the QIAamp® viral RNA Mini Kit (QIAGEN). First strand cDNA was made using random hexamer primer and the RNA LA PCR™ (AMV) Kit (TaKaRa), and subjected to amplification using a nested PCR. The first PCR was performed using primers VNUP 5′GATAA TGGAC CCCAA TCAAA CCAA3′ (SEQ ID NO:3) and VNLOW 5′CTGAG TTGAA TCAGG AGAAG CTCC3′ (SEQ ID NO:4), and the second PCR with primers N355UP 5′GAACT GGCCC AGAAG CTTCA CT3′ (SEQ ID NO:5) and N355LOW 5′TTGGC CTTTA CCAGA AACTT TG3′ (SEQ ID NO:6). The size of the nested PCR product was 355 bp. All PCR products were confirmed by nucleotide sequencing.

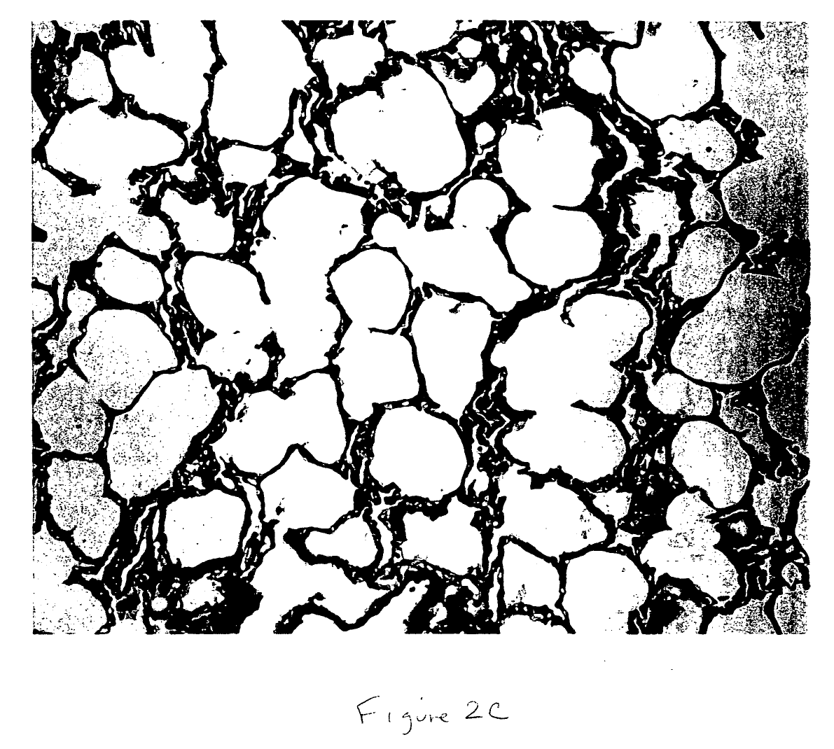

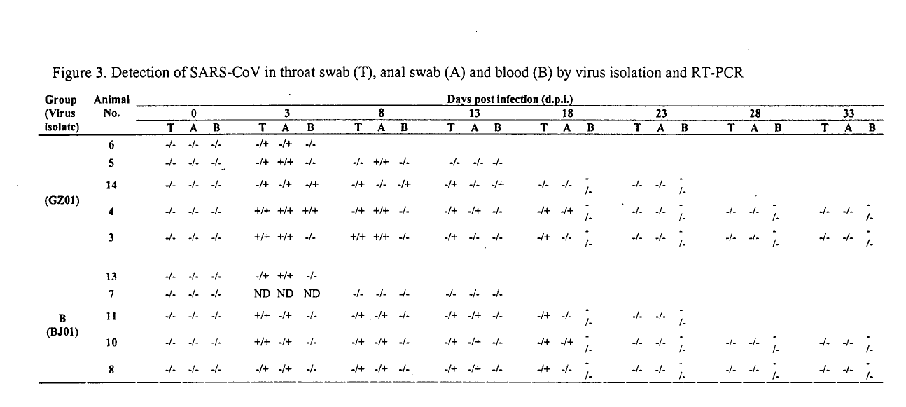

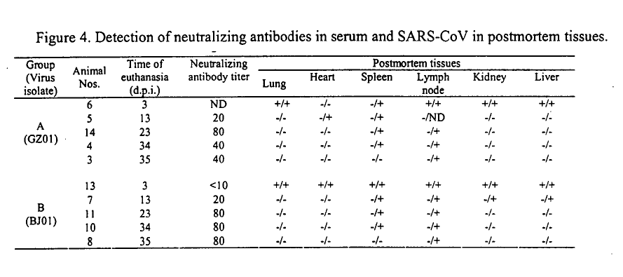

Results presented in FIG. 3 indicated that viral genome was detected by RT-PCR in throat and anal swabs from days 3-18 p.i. and live virus was isolated on day 3 p.i from animals in both groups, and on day 8 p.i. from animals in group A only (FIG. 3). Morphologically, the virus recovered was identical to that used for inoculation (data not shown). The detection frequency of virus in blood samples was very low, with only one animal (No. 4) in group A giving positive results for both virus isolation and RT-PCR on day 3 p.i., and a second animal (No. 14) in group A showing positive RT-PCR for samples taken on days 3, 8 and 13 p.i. In contrast, virus was readily detected up to day 13 p.i. in a variety of organs, including lung, liver, kidney, and heart (FIG. 4). Virus was still detected by RT-PCR at the end of the experiment (34 to 35 d.p.i.) in lymph nodes and spleen.

Antibody Detection

For antibody detection, 2-fold dilutions of serum were tested in a microneutralization assay for the presence of antibodies that neutralized the infectivity of 200 TCID₅₀ of SARS-CoV in Vero E6 cell monolayers, with four wells per dilution on a 96-well plate. The presence of CPE was read on days 3 and 4, and neutralizing titers determined from the dilution factor of serum that completely prevented CPE in 50% of the wells. Serum samples taken on the day of euthanasia were analyzed, and neutralizing antibodies were detected in samples taken from 13 d.p.i. onwards, with the antibody titers varying from 20 at 13 d.p.i. to 80 at 34 or 35 d.p.i. (FIG. 4).

Conclusion

The focus of this Example was to test and compare the susceptibility of farmed civets to two different exogenous human SARS-CoV strains, the early-phase strain GZ01 and the middle-phase strain BJ01. The results of this Example conclusively show that civets are readily susceptible to experimental infection by both of these strains of human SARS-CoV, a particularly novel result in light of the divergence of early-stage and particularly middle-stage human SARS-CoV strains from the civet SARS-CoV-like strains that have been shown to exist in civets in the wild.

This Example further demonstrates that civets infected with exogenous human SARS-CoV strains develop SARS-like symptoms, as judged by clinical, virological, and serological evidence. Interestingly, middle-stage strain BJ01 appears from the data to produce a higher average body temperature (FIG. 1A) and slightly stronger antibody response (FIG. 4) than early-stage strain GZ01. This result is consistent with the correlation of later staging with better adaptation of the SARS-CoV strain to the human host. More data will extend this observation to other early-, middle-, and late-stage human SARS-CoV strains, i.e., might allow for the correlation of adaptation to humans with the degree of SARS-like symptoms exhibited.

Also of interest is the fact that viral genomic RNA may be detected in spleen and lymph nodes up to 34/35 days p.i. (FIG. 4), suggesting that these tissues may be able to support persistent infection of SARS-CoV. Thus the present invention contemplates experiments focused on these tissues, and particularly on the possible role of these tissues in such persistent infections.

### EXAMPLE 2

### Determination of Human SARS-CoV Coronavirus Dosage and Path of Administration Required for Infection of Civets

Given the novel results of Example 1 demonstrating that civets may be infected with exogenous coronaviral strains to produce SARS-like symptoms, and, even more surprisingly, that such SARS-like symptoms occur after infection with early- and middle-stage human SARS-CoV coronaviral strains, it will be advantageous to determine the dosages of exogenous coronavirus required to elicit such SARS-like symptoms, as well as what paths of administration are effective for producing such symptoms.

Thus symptom-free civets obtained as in Example 1 are infected with, e.g., varying dosages of a human-derived SARS-CoV strain or variant thereof, for example 10⁵ TCID₅₀, 10⁴ TCID₅₀, 10³ TCID₅₀, 10² TCID₅₀, 10¹ TCID₅₀ of the selected coronaviral strain. In one set of experiments these infections are done intratracheally. Infection may also be done via routes most likely to serve for transmission of the coronavirus in the wild, e.g., intranasally, intraorally, or subcutaneously.

Post-infection these animals are analyzed at regular intervals for SARS-like symptoms as described above, e.g., clinical symptoms, viral load, tissue abnormalities/damage, etc. For example, in one protocol animals are subjected to temperature measuring, clinical signs observation and leukocytes counting at daily intervals, with pharyngeal and rectal swabs and blood samples taken every three days post-infection for virus isolation and real-time RT-PCR detections.

Animals are sacrificed at a suitable period post-infection, e.g., 21 days post-infection, and subjected to pathological examinations. In one protocol, for example, brain, lung, spleen, lymph nodes, kidney and liver are taken for virus isolation, real-time RT-PCR, and histopathological examinations. In various protocols blood samples are subjected to serum neutralization analysis.

These protocols will allow the determination of the minimum dosage of human-derived SARS-CoV strain or variant thereof required to elicit SARS-like symptoms when administered by a particular route of infection.

### EXAMPLE 3

### Determination of Ability of Human SARS-CoV Coronavirus-Infected Civets to Infect Non-Infected Civets

In both the 2002-2003 and 2003-2004 SARS epidemics it was shown that humans were able to spread the SARS-CoV coronavirus to other humans, a process that presumably also occurs in wild civets transmitting SARS-CoV-like coronavirus to civets lacking this coronavirus.

In order to better understand the nature of animal-animal transmission, civets exhibiting SAR-like symptoms after infection with human-derived SARS-CoV strains as described in, e.g., Examples 1 or 2 above are placed in proximity with symptom-free animals for varying times, and the appearance of SARS-like symptoms in the originally healthy animals are assayed using any of the methods described previously. These experiments include protocols in which “proximity” includes adjacent cages; also included are protocols in which cages are more distant but air supply between cages is shared, i.e., protocols in which transmission via, e.g., aerosols, is examined. Protocols also contemplated include those in which healthy animals are exposed to the feces, urine, or other body fluids of infected animals expressing SARS-like symptoms. Additionally, all these protocols will assay various stages of progression of the SARS-like symptoms in the infected animals in determining transmission; that is, it will be important to determine whether particular stages in the progression of the disease in infected animals elicit a higher degree of transmission than other stages.

### EXAMPLE 4

### Determination of Civet SARS-CoV-Like Coronavirus Dosage and Path of Administration Required for Infection of Civets

Although efforts to prevent SARS in humans have focused on the development of human vaccines, because available evidence suggest that the disease is transmitted to humans from civets either as the civet-derived SARS-CoV-like coronovirus or related coronavirus, information regarding the nature of infection of civets by this civet SARS-CoV-like coronaviral sequence may lead to prevention of the disease in civets, and therefore minimized transmission to humans. Thus in this Example, dosage and path of administration for infection of civets with civet SARS-CoV-like coronavirus strains will be determined.

Thus symptom-free civets obtained as in Example 1 are infected with, e.g., varying dosages of a civet-derived SARS-CoV-like strain or variant thereof, for example 10⁵ TCID₅₀, 10⁴ TCID₅₀, 10³ TCID₅₀, 10² TCID₅₀, 10¹ TCID₅₀ of the selected coronaviral strain. In one set of experiments these infections are done intratracheally. Infection may also be done via routes most likely to serve for transmission of the coronavirus in the wild, e.g., intranasally, intraorally, or subcutaneously.

Post-infection these animals are analyzed at regular intervals for SARS-like symptoms as described above, e.g., clinical symptoms, viral load, tissue abnormalities/damage, etc. For example, in one protocol animals are subjected to temperature measuring, clinical signs observation and leukocytes counting at daily intervals, with pharyngeal and rectal swabs and blood samples taken every three days post-infection for virus isolation and real-time RT-PCR detections.

Animals are sacrificed at a suitable period post-infection, e.g., 21 days post-infection, and subjected to pathological examinations. In one protocol, for example, brain, lung, spleen, lymph nodes, kidney and liver are taken for virus isolation, real-time RT-PCR, and histopathological examinations. In various protocols blood samples are subjected to serum neutralization analysis.

These protocols will allow the determination of the minimum dosage of civet-derived SARS-CoV-like strain or variant thereof required to elicit SARS-like symptoms when administered by a particular route of infection.

### EXAMPLE 5

### Vaccine Development

As discussed previously, the development of vaccines in the civet animal model system of the invention is based on the use of whole animals or on in vitro assays using cultured civet cells to determine the protective effects of various putative coronaviral vaccine agents against one or more human-derived SARS-CoV coronaviral strains or civet-derived SARS-CoV-like coronaviral-infected strains of the present invention, where such putative coronaviral vaccine agents include, but are not limited to: agents based on whole-coronaviruses or variants thereof, including live-attenuated and inactivated coronaviruses; components of the coronavirus, e.g., the S-protein; and, agents based on antibodies against the coronavirus, or component or components thereof.

In a whole animal model for vaccine development, a civet is treated with one or more putative coronaviral vaccine agents and then, after a suitable time delay, is infected with exogenous coronavirus and monitored for SARS-like symptoms. Thus for example symptom-free civets obtained as in Example 1 are treated with a putative coronaviral vaccine agent and then infected with, e.g., varying dosages of a human-derived SARS-CoV strain or variant thereof, via a selected route of infection, e.g., intratracheally, intranasally, intraorally, subcutaneously, etc.

Post-infection these animals are analyzed at regular intervals for SARS-like symptoms as described above, e.g., clinical symptoms, viral load, tissue abnormalities/damage, etc. For example, in one protocol animals are subjected to temperature measuring, clinical signs observation and leukocytes counting at daily intervals, with pharyngeal and rectal swabs and blood samples taken every three days post-infection for virus isolation and real-time RT-PCR detections.

Animals are sacrificed at a suitable period post-infection, e.g., 21 days post-infection, and subjected to pathological examinations. In one protocol, for example, brain, lung, spleen, lymph nodes, kidney and liver are taken for virus isolation, real-time RT-PCR, and histopathological examinations. In various protocols blood samples are subjected to serum neutralization analysis.

These protocols will allow the determination of the effect of a putative coronaviral vaccine agent on the protection of a infected civet from one or more of the SARS-like symptoms that untreated civets exhibit post-infection.

While the present invention has been described with reference to its preferred embodiments, one of one of ordinary skill in the relevant art will understand that the present invention is not intended to be limited by these preferred embodiments, and is instead contemplated to include all embodiments consistent with the spirit and scope of the present invention as defined by the appended claims. The entire disclosures of all references, applications, patents, and publications cited herein are hereby incorporated by reference.

What is claimed is:

1. A civet infected with an exogenous coronavirus or variant or derivative of said exogenous coronavirus.

2. The civet of claim 1, wherein said exogenous coronavirus is a human-derived exogenous coronavirus, or variant or derivative thereof.

3. The civet of claim 2, wherein said human-derived exogenous coronavirus is selected from the group consisting of an early-stage human-derived exogenous coronavirus, a middle-stage human-derived exogenous coronavirus, and a late-stage human-derived exogenous coronavirus.

4. The civet of claim 3, wherein said human-derived exogenous coronavirus is an early-stage human-derived exogenous coronavirus.

5. The civet of claim 4, wherein said early-stage human-derived exogenous coronavirus is selected from the group consisting of GZ02, GZ01, HGZ8L1-A, HSZ-Cc, HSZ-A, HSZ-Bb, HSZ-Cb, HSZ-Bc, GZ50, GZ-A, JMD, HGZ8L1-B, ZS-A, ZS-B, and ZS-C.

6. The civet of claim 5, wherein said early-stage human-derived exogenous coronavirus is GZ01.

7. The civet of claim 3, wherein said human-derived exogenous coronavirus is a middle-stage human-derived exogenous coronavirus.

8. The civet of claim 7, wherein said middle-stage human-derived exogenous coronavirus is selected from the group consisting of BJ04, BJ03, BJ02, BJ01, CUHK-W1, HZS2-D, HZS2-E, HZS2-C, HGZ8L2, HZS2-Bb, HSZ2-A, HZS2-Fc, and HZS2-Fb.

9. The civet of claim 8, wherein said middle-stage human-derived exogenous coronavirus is BJ01.

10. The civet of claim 3, wherein said human-derived exogenous coronavirus is a late-stage human-derived exogenous coronavirus.

11. The civet of claim 10, wherein said late-stage human-derived exogenous coronavirus is selected from the group consisting of TWC, Sin2679, ZJ01, HSR, TW1, HKU-39849, GZ-D, Urbani, Sin2748, Sin2677, Sin2500, Frankfurt, Sin2774, CUHK-Su10, CUHK-LC1, CUHK-AG01, CUHK-AG02, CUHK-AG03, TWH, TC1, TWY, TWS, TWK, TWJ, TC3, TC2, GZ-B, GZ-C, TOR2, CUHK-LC2, CUHK-LC3, CUHK-LC4, and CUHK-LC5.

12. The civet of claim 1, wherein said exogenous coronavirus is a civet-derived exogenous coronavirus, or variant or derivative thereof.

13. Isolated cells of the civet of claim 2.

14. Isolated coronavirus of the civet of claim 2.

15. Isolated genomic RNA of the coronavirus of claim 14.

16. A method for producing coronavirus in a civet, comprising infecting a civet with an exogenous coronavirus or variant or derivative of said exogenous coronavirus.

17. The method of claim 16, wherein said exogenous coronavirus is a human-derived exogenous coronavirus.

18. The method of claim 17, wherein said human-derived exogenous coronavirus is GZ01 or BJ01.

19. A method for assaying the development of SARS-like symptoms in a civet, comprising:
a) infecting a civet with an exogenous coronavirus; and,
b) assaying the development of said SARS-like symptoms in said civet.

20. A method for assaying the effect of a putative coronaviral protection agent on the development of SARS-like symptoms in a civet, comprising:
a) introducing a putative coronaviral protection agent into a civet;
b) infecting said civet with an exogenous coronavirus; and,
c) assaying the development of said SARS-like symptoms in said civet.

21. The method of claim 20, wherein said putative coronaviral protection agent is selected from the group consisting of an isolated human-derived coronaviral protein, an early stage human-derived coronavirus, an antibody against human-derived SARS viral protein S, and a killed human-derived SARS virus.

22. A method for assaying the effect of a putative coronaviral vaccine agent on the development of SARS-like symptoms in a civet, comprising:
a) introducing a putative coronaviral vaccine agent into a civet;
b) infecting said civet with an exogenous coronavirus; and,
c) assaying the development of said SARS-like symptoms in said civet.

23. A method for preventing or reducing SARS-like symptoms in a civet, comprising inoculating a civet with a coronaviral protection agent or coronaviral vaccine agent.


SEQUENCE LISTING (6 sequences)

SEQ ID NO: 1 - DNA, 29757 bp, SARS coronavirus
tacccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcactgccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagagagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgcagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacaccttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaggactgctcttaagaaatgcaaatctgcatcttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttaaccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaatttttgcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctatgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagttcccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttacatcgttatgccagtacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgttgagagcaatcaatggtgacttcttgcattttctacctcgtgtttttagtgctgttggcaacatttgctacacaccttccaaactcattgagtatagtgattttgctacctctgcttgcgttcttgctgcagagtgtacaatttttaaggatgctatgggcaaacctgtgccatattgttatgacactaatttgctagagggttctatttcttatagtgagcttcgtccagacactcgttatgtgcttatggatggttccatcatacagtttcctaacatttacctggagggttctgttagagtagtaacaacttttgatgctgagtactgtagacatggtacatgcgaaaggtcagaagcaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgcttttggtgagtacaaccatgttgttgctgctaatgcacttttgtttttgatgtctttcactatactctgtctggcaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagccttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaaggatgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcggttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataaccttaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaacgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcatcaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttatcctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtgtctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttaatgacttcgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgaattcgtctccgacgcagattctactttaattggagactgtgcaacagtacatacggctaataaatgggaccttatcattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaagacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaataactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacccatttttaagttgcctcttggtattaacattacaaattttagagccattcttacagcctttttacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaagagatgttgtgagattccctaatattacaaacttgtgtccttttggagaggtttttaatgctactaaattcccttctgtctatgcatgggagaggaaaagaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttatgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaagattttggtggttttaatttttcacaaatattacctgaccctctaaagtcaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgcggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatccaagaacccattactttatgatgccaactactttgtttgctggcacacacataactatgactactgtataccatataacagtgtcacagatacaattgtcgttactgcaggtgacggcatttcaacaccaaaactcaaagaagactaccaaattggtggttattctgaggattggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgcaaatccagcaatggatccaatttatgatgagccgacgacgactactagcgtgcctttgtaagcacaagaaagtgagtacgaacttatgtactcattcgtttcggaagaaacaggtacgttaatagttaatagcgtacttctttttcttgctttcatggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacttatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatacgcactgtagtacagcgctgtgcatctaataaacctcatgtgcttgaagatccttgtcctactggttaccaacctgaatggaatataaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtatttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaa

SEQ ID NO: 2 - DNA, 29725 bp, SARS coronavirus
ccaggaaaagccaaccaacctcgatctcttgtagatctgttctctaaacgaactttaaaatctgtgtagctgtcgctcggctgcatgcctagtgcacctacgcagtataaacaataataaattttactgtcgttgacaagaaacgagtaactcgtccctcttctgcagactgcttacggtttcgtccgtgttgcagtcgatcatcagcatacctaggtttcgtccgggtgtgaccgaaaggtaagatggagagccttgttcttggtgtcaacgagaaaacacacgtccaactcagtttgcctgtccttcaggttagagacgtgctagtgcgtggcttcggggactctgtggaagaggccctatcggaggcacgtgaacacctcaaaaatggcacttgtggtctagtagagctggaaaaaggcgtactgccccagcttgaacagccctatgtgttcattaaacgttctgatgccttaagcaccaatcacggccacaaggtcgttgagctggttgcagaaatggacggcattcagtacggtcgtagcggtataacactgggagtactcgtgccacatgtgggcgaaaccccaattgcataccgcaatgttcttcttcgtaagaacggtaataagggagccggtggtcatagctatggcatcgatctaaagtcttatgacttaggtgacgagcttggcactgatcccattgaagattatgaacaaaactggaacactaagcatggcagtggtgcactccgtgaactcactcgtgagctcaatggaggtgcagtcactcgctatgtcgacaacaatttctgtggcccagatgggtaccctcttgattgcatcaaagattttctcgcacgcgcgggcaagtcaatgtgcactctttccgaacaacttgattacatcgagtcgaagagaggtgtctactgctgccgtgaccatgagcatgaaattgcctggttcactgagcgctctgataagagctacgagcaccagacacccttcgaaattaagagtgccaagaaatttgacactttcaaaggggaatgcccaaagtttgtgtttcctcttaactcaaaagtcaaagtcattcaaccacgtgttgaaaagaaaaagactgagggtttcatggggcgtatacgctctgtgtaccctgttgcatctccacaggagtgtaacaatatgcacttgtctaccttgatgaaatgtaatcattgcgatgaagtttcatggcagacgtgcgactttctgaaagccacttgtgaacattgtggcactgaaaatttagttattgaaggacctactacatgtgggtacctacctactaatgctgtagtgaaaatgccatgtcctgcctgtcaagacccagagattggacctgagcatagtgttgcagattatcacaaccactcaaacattgaaactcgactccgcaagggaggtaggactagatgttttggaggctgtgtgtttgcctatgttggctgctataataagcgtgcctactgggttcctcgtgctagtgctgatattggctcaggccatactggcattactggtgacaatgtggagaccttgaatgaggatctccttgagatactgagtcgtgaacgtgttaacattaacattgttggcgattttcatttgaatgaagaggttgccatcattttggcatctttctctgcttctacaagtgcctttattgacactataaagagtcttgattacaagtctttcaaaaccattgttgagtcctgcggtaactataaagttaccaagggaaagcccgtaaaaggtgcttggaacattggacaacagagatcagttttaacaccactgtgtggttttccctcacaggctgctggtgttatcagatcaatttttgcgcgcacacttgatgcagcaaaccactcaattcctgatttgcaaagagcagctgtcaccatacttgatggtatttctgaacagtcattacgtcttgtcgacgccatggtttatacttcagacctgctcaccaacagtgtcattattatggcatatgtaactggtggtcttgtacaacagacttctcagtggttgtctaatcttttgggcactactgttgaaaaactcaggcctatctttgaatggattgaggcgaaacttagtgcaggagttgaatttctcaaggatgcttgggagattctcaaatttctcattacaggtgtttttgacatcgtcaagggtcaaatacaggttgcttcagataacatcaaggattgtgtaaaatgcttcattgatgttgttaacaaggcactcgaaatgtgcattgatcaagtcactatcgctggcgcaaagttgcgatcactcaacttaggtgaagtcttcatcgctcaaagcaagggactttaccgtcagtgtatacgtggcaaggagcagctgcaactactcatgcctcttaaggcaccaaaagaagtaacctttcttgaaggtgattcacatgacacagtacttacctctgaggaggttgttctcaagaacggtgaactcgaagcactcgagacgcccgttgatagcttcacaaatggagctatcgttggcacaccagtctgtgtaaatggcctcatgctcttagagattaaggacaaagaacaatactgcgcattgtctcctggtttactggctacaaacaatgtctttcgcttaaaagggggtgcaccaattaaaggtgtaacctttggagaagatactgtttgggaagttcaaggttacaagaatgtgagaatcacatttgagcttgatgaacgtgttgacaaagtgcttaatgaaaagtgctctgtctacactgttgaatccggtaccgaagttactgagtttgcatgtgttgtagcagaggctgttgtgaagactttacaaccagtttctgatctccttaccaacatgggtattgatcttgatgagtggagtgtagctacattctacttatttgatgatgctggtgaagaaaacttttcatcacgtatgtattgttccttttaccctccagatgaggaagaagaggacgatgcagagtgtgaggaagaagaaattgatgaaacctgtgaacatgagtacggtacagaggatgattatcaaggtctccctctggaatttggtgcctcagctgaaacagttcgagttgaggaagaagaagaggaagactggctggatgatactactgagcaatcagagattgagccagaaccagaacctacacctgaagaaccagttaatcagtttactggttatttaaaacttactgacaatgttgccattaaatgtgttgacatcgttaaggaggcacaaagtgctaatcctatggtgattgtaaatgctgctaacatacacctgaaacatggtggtggtgtagcaggtgcactcaacaaggcaaccaatggtgccatgcaaaaggagagtgatgattacattaagctaaatggccctcttacagtaggagggtcttgtttgctttctggacataatcttgctaagaagtgtctgcatgttgttggacctaacctaaatgcaggtgaggacatccagcttcttaaggcagcatatgaaaatttcaattcacaggacatcttacttgcaccattgttgtcagcaggcatatttggtgctaaaccacttcagtctttacaagtgtgcgtgcagacggttcgtacacaggtttatattgcagtcaatgacaaagctctttatgagcaggttgtcatggattatcttgataacctgaagcctagagtggaagcacctaaacaagaggagccaccaaacacagaagattccaaaactgaggagaaatctgtcgtacagaagcctgtcgatgtgaagccaaaaattaaggcctgcattgatgaggttaccacaacactggaagaaactaagtttcttaccaataagttactcttgtttgctgatatcaatggtaagctttaccatgattctcagaacatgcttagaggtgaagatatgtctttccttgagaaggatgcaccttacatggtaggtgatgttatcactagtggtgatatcacttgtgttgtaataccctccaaaaaggctggtggcactactgagatgctctcaagagctttgaagaaagtgccagttgatgagtatataaccacgtaccctggacaaggatgtgctggttatacacttgaggaagctaagactgctcttaagaaatgcaaatctgcattttatgtactaccttcagaagcacctaatgctaaggaagagattctaggaactgtatcctggaatttgagagaaatgcttgctcatgctgaagagacaagaaaattaatgcctatatgcatggatgttagagccataatggcaaccatccaacgtaagtataaaggaattaaaattcaagagggcatcgttgactatggtgtccgattcttcttttatactagtaaagagcctgtagcttctattattacgaagctgaactctctaaatgagccgcttgtcacaatgccaattggttatgtgacacatggttttaatcttgaagaggctgcgcgctgtatgcgttctcttaaagctcctgccgtagtgtcagtatcatcaccagatgctgttactacatataatggatacctcacttcgtcatcaaagacatctgaggagcactttgtagaaacagtttctttggctggctcttacagagattggtcctattcaggacagcgtacagagttaggtgttgaatttcttaagcgtggtgacaaaattgtgtaccacactctggagagccccgtcgagtttcatcttgacggtgaggttctttcacttgacaaactaaagagtctcttatccctgcgggaggttaagactataaaagtgttcacaactgtggacaacactaatctccacacacagcttgtggatatgtctatgacatatggacagcagtttggtccaacatacttggatggtgctgatgttacaaaaattaaacctcatgtaaatcatgagggtaagactttctttgtactacctagtgatgacacactacgtagtgaagctttcgagtactaccatactcttgatgagagttttcttggtaggtacatgtctgctttaaaccacacaaagaaatggaaatttcctcaagttggtggtttaacttcaattaaatgggctgataacaattgttatttgtctagtgttttattagcacttcaacagcttgaagtcaaattcaatgcaccagcacttcaagaggcttattatagagcccgtgctggtgatgctgctaacttttgtgcactcatactcgcttacagtaataaaactgttggcgagcttggtgatgtcagagaaactatgacccatcttctacagcatgctaatttggaatctgcaaagcgagttcttaatgtggtgtgtaaacattgtggtcagaaaactactaccttaacgggtgtagaagctgtgatgtatatgggtactctatcttatgataatcttaagacaggtgtttccattccatgtgtgtgtggtcgtgatgctacacaatatctagtacaacaagagtcttcttttgttatgatgtctgcaccacctgctgagtataaattacagcaaggtacattcttatgtgcgaatgagtacactggtaactatcagtgtggtcattacactcatataactgctaaggagaccctctatcgtattgacggagctcaccttacaaagatgtcagagtacaaaggaccagtgactgatgttttctacaaggaaacatcttacactacaaccatcaagcctgtgtcgtataaactcgatggagttacttacacagagattgaaccaaaattggatgggtattataaaaaggataatgcttactatacagagcagcctatagaccttgtaccaactcaaccattaccaaatgcgagttttgataatttcaaactcacatgttctaacacaaaatttgctgatgatttaaatcaaatgacaggcttcacaaagccagcttcacgagagctatctgtcacattcttcccagacttgaatggcgatgtagtggctattgactatagacactattcagcgagtttcaagaaaggtgctaaattactgcataagccaattgtttggcacattaaccaggctacaaccaagacaacgttcaaaccaaacacttggtgtttacgttgtctttggagtacaaagccagtagatacttcaaattcatttgaagttctggcagtagaagacacacaaggaatggacaatcttgcttgtgaaagtcaacaacccacctctgaagaagtagtggaaaatcctaccatacagaaggaagtcatagagtgtgacgtgaaaactaccgaagttgtaggcaatgtcatacttaaaccatcagatgaaggtgttaaagtaacacaagagttaggtcatgaggatcttatggctgcttatgtggaaaacacaagcattaccattaagaaacctaatgagctttcactagccttaggtttaaaaacaattgccactcatggtattgctgcaattaatagtgttccttggagtaaaattttggcttatgtcaaaccattcttaggacaagcagcaattacaacatcaaattgcgctaagagattagcacaacgtgtgtttaacaattatatgccttatgtgtttacattattgttccaattgtgtacttttactaaaagtaccaattctagaattagagcttcactacctacaactattgctaaaaatagtgttaagagtgttgctaaattatgtttggatgccggcattaattatgtgaagtcacccaaattttctaaattgttcacaatcgctatgtggctattgttgttaagtatttgcttaggttctctaatctgtgtaactgctgcttttggtgtactcttatctaattttggtgctccttcttattgtaatggcgttagagaattgtatcttaattcgtctaacgttactactatggatttctgtgaaggttcttttccttgcagcatttgtttaagtggattagactcccttgattcttatccagctcttgaaaccattcaggtgacgatttcatcgtacaagctagacttgacaattttaggtctggccgctgagtgggttttggcatatatgttgttcacaaaattcttttatttattaggtctttcagctataatgcaggtgttctttggctattttgctagtcatttcatcagcaattcttggctcatgtggtttatcattagtattgtacaaatggcacccgtttctgcaatggttaggatgtacatcttctttgcttctttctactacatatggaagagctatgttcatatcatggatggttgcacctcttcgacttgcatgatgtgctataagcgcaatcgtgccacacgcgttgagtgtacaactattgttaatggcatgaagagatctttctatgtctatgcaaatggaggccgtggcttctgcaagactcacaattggaattgtctcaattgtgacacattttgcactggtagtacattcattagtgatgaagttgctcgtgatttgtcactccagtttaaaagaccaatcaaccctactgaccagtcatcgtatattgttgatagtgttgctgtgaaaaatggcgcgcttcacctctactttgacaaggctggtcaaaagacctatgagagacatccgctctcccattttgtcaatttagacaatttgagagctaacaacactaaaggttcactgcctattaatgtcatagtttttgatggcaagtccaaatgcgacgagtctgcttctaagtctgcttctgtgtactacagtcagctgatgtgccaacctattctgttgcttgaccaagctcttgtatcagacgttggagatagtactgaagtttccgttaagatgtttgatgcttatgtcgacaccttttcagcaacttttagtgttcctatggaaaaacttaaggcacttgttgctacagctcacagcgagttagcaaagggtgtagctttagatggtgtcctttctacattcgtgtcagctgcccgacaaggtgttgttgataccgatgttgacacaaaggatgttattgaatgtctcaaactttcacatcactctgacttagaagtgacaggtgacagttgtaacaatttcatgctcacctataataaggttgaaaacatgacgcccagagatcttggcgcatgtattgactgtaatgcaaggcatatcaatgcccaagtagcaaaaagtcacaatgtttcactcatctggaatgtaaaagactacatgtctttatctgaacagctgcgtaaacaaattcgtagtgctgccaagaagaacaacataccttttagactaacttgtgctacaactagacaggttgtcaatgtcataactactaaaatctcactcaagggtggtaagattgttagtacttgttttaaacttatgcttaaggccacattattgtgcgttcttgctgcattggtttgttatatcgttatgccattacatacattgtcaatccatgatggttacacaaatgaaatcattggttacaaagccattcaggatggtgtcactcgtgacatcatttctactgatgattgttttgcaaataaacatgctggttttgacgcatggtttagccagcgtggtggttcatacaaaaatgacaaaagctgccctgtagtagctgctatcattacaagagagattggtttcatagtgcctggcttaccgggtactgtgctgagagcaatcaatggtgacttcttgcattttctacctcgtgtttttagtgctgttggcaacatttgctacacaccttccaaactcattgagtatagtgattttgctacctctgcttgcgttcttgctgctgagtgtacaatttttaaggatgctatgggcaaacctgtgccatattgttatgacactaatttgctagagggttctatttcttatagtgagcttcgtccagacactcgttatgtgcttatggatggttccatcatacagtttcctaacacttacctggagggttctgttagagtagtaacaacttttgatgctgagtactgtagacatggtacatgcgaaaggtcagaagtaggtatttgcctatctaccagtggtagatgggttcttaataatgagcattacagagctctatcaggagttttctgtggtgttgatgcgatgaatctcatagctaacatctttactcctcttgtgcaacctgtgggtgctttagatgtgtctgcttcagtagtggctggtggtattattgccatattggtgacttgtgctgcctactactttatgaaattcagacgtgcttttggtgagtacaaccatgttgttgctgctaatgcacttttgtttttgatgtctttcactatactctgtctggtaccagcttacagctttctgccgggagtctactcagtcttttacttgtacttgacattctatttcaccaatgatgtttcattcttggctcaccttcaatggtttgccatgttttctcctattgtgcctttttggataacagcaatctatgtattctgtatttctctgaagcactgccattggttctttaacaactatcttaggaaaagagtcatgtttaatggagttacatttagtaccttcgaggaggctgctttgtgtacctttttgctcaacaaggaaatgtacctaaaattgcgtagcgagacactgttgccacttacacagtataacaggtatcttgctctatataacaagtacaagtatttcagtggagtcttagatactaccagctatcgtgaagcagcttgctgccacttagcaaaggctctaaatgactttagcaactcaggtgctgatgttctctaccaaccaccacagacatcaatcacttctgctgttctgcagagtggttttaggaaaatggcattcccgtcaggcaaagttgaagggtgcatggtacaagtaacctgtggaactacaactcttaatggattgtggttggatgacacagtatactgtccaagacatgtcatttgcacagcagaagacatgcttaatcctaactatgaagatctgctcattcgcaaatccaaccatagctttcttgttcaggctggcaatgttcaacttcgtgttattggccattctatgcaaaattgtctgcttaggcttaaagttgatacttctaaccctaagacacccaagtataaatttgtccgtatccaacctggtcaaacattttcagttctagcatgctacaatggttcaccatctggtgtttatcagtgtgccatgagacctaatcataccattaaaggttctttccttaatggatcatgtggtagtgttggttttaacattgattatgattgcgtgtctttctgctatatgcatcatatggagcttccaacaggagtacacgctggtactgacttagaaggtaaattctatggtccatttgttgacagacaaactgcacaggctgcaggtacagacacaaccataaccttaaatgttttggcatggctgtatgctgctgttatcaatggtgataggtggtttcttaatagattcaccactactttgaatgactttaaccttgtggcaatgaagtacaactatgaacctttgacacaagatcatgttgacatattgggacctctttctgctcaaacaggaattgccgtcttagatatgtgtgctgctttgaaagagctgctgcagaatggtatgaatggtcgtactatccttggtagcactattttagaagatgagtttacaccatttgatgttgttagacaatgctctggtgttaccttccaaggtaagttcaagaaaattgttaagggcactcatcattggatgcttttaactttcttgacatcactattgattcttgttcaaagtacacagtggtcactgtttttctttgtttacgagaatgctttcttgccatttactcttggtattatggcaattgctgcatgtgctatgctgcttgttaagcataagcacgcattcttgtgcttgtttctgttaccttctcttgcaacagttgcttactttaatatggtctacatgcctgctagctgggtgatgcgtatcatgacatggcttgaattggctgacactagcttgtctggttataggcttaaggattgtgttatgtatgcttcagctttagttttgcttattctcatgacagctcgcactgtttatgatgatgctgctagacgtgtttggacactgatgaatgtcattacacttgtttacaaagtctactatggtaatgctttagatcaagctatttccatgtgggccttagttatttctgtaacctctaactattctggtgtcgttacgactatcatgtttttagctagagctatagtgtttgtgtgtgttgagtattacccattgttatttattactggcaacaccttacagtgtatcatgcttgtttattgtttcttaggctattgttgctgctgctactttggccttttctgtttactcaaccgttacttcaggcttactcttggtgtttatgactacttggtctctacacaagaatttaggtatatgaactcccaggggcttttgcctcctaagagtagtattgatgctttcaagcttaacattaagttgttgggtattggaggtaaaccatgtatcaaggttgctactgtacagtctaaaatgtctgacgtaaagtgcacatctgtggtactgctctcggttcttcaacaacttagagtagagtcatcttctaaattgtgggcacaatgtgtacaactccacaatgatattcttcttgcaaaagacacaactgaagctttcgagaagatggtttctcttttgtctgttttgctatccatgcagggtgctgtagacattaataggttgtgcgaggaaatgctcgataaccgtgctactcttcaggctattgcttcagaatttagttctttaccatcatatgccgcttatgccactgcccaggaggcctatgagcaggctgtagctaatggtgattctgaagtcgttctcaaaaagttaaagaaatctttgaatgtggctaaatctgagtttgaccgtgatgctgccatgcaacgcaagttggaaaagatggcagatcaggctatgacccaaatgtacaaacaggcaagatctgaggacaagagggcaaaagtaactagtgctatgcaaacaatgctcttcactatgcttaggaagcttgataatgatgcacttaacaacattatcaacaatgcgcgtgatggttgtgttccactcaacatcataccattgactacagcagccaaactcatggttgttgtccctgattatggtacctacaagaacacttgtgatggtaacacctttacatatgcatctgcactctgggaaatccagcaagttgttgatgcggatagcaagattgttcaacttagtgaaattaacatggacaattcaccaaatttggcttggcctcttattgttacagctctaagagccaactcagctgttaaactacagaataatgaactgagtccagtagcactacgacagatgtcctgtgcggctggtaccacacaaacagcttgtactgatgacaatgcacttgcctactataacaattcgaagggaggtaggtttgtgctggcattactatcagaccaccaagatctcaaatgggctagattccctaagagtgatggtacaggtacaatttacacagaactggaaccaccttgtaggtttgttacagacacaccaaaagggcctaaagtgaaatacttgtacttcatcaaaggcttaaacaacctaaatagaggtatggtgctgggcagtttagctgctacagtacgtcttcaggctggaaatgctacagaagtacctgccaattcaactgtgctttccttctgtgcttttgcagtagaccctgctaaagcatataaggattacctagcaagtggaggacaaccaatcaccaactgtgtgaagatgttgtgtacacacactggtacaggacaggcaattactgtaacaccagaagctaacatggaccaagagtcctttggtggtgcttcatgttgtctgtattgtagatgccacattgaccatccaaatcctaaaggattctgtgacttgaaaggtaagtacgtccaaatacctaccacttgtgctaatgacccagtgggttttacacttagaaacacagtctgtaccgtctgcggaatgtggaaaggttatggctgtagttgtgaccaactccgcgaacccttgatgcagtctgcggatgcatcaacgtttttaaacgggtttgcggtgtaagtgcagcccgtcttacaccgtgcggcacaggcactagtactgatgtcgtctacagggcttttgatatttacaacgaaaaagttgctggttttgcaaagttcctaaaaactaattgctgtcgcttccaggagaaggatgaggaaggcaatttattagactcttactttgtagttaagaggcatactatgtctaactaccaacatgaagagactatttataacttggttaaagattgtccagcggttgctgtccatgactttttcaagtttagagtagatggtgacatggtaccacatatatcacgtcagcgtctaactaaatacacaatggctgatttagtctatgctctacgtcattttgatgagggtaattgtgatacattaaaagaaatactcgtcacatacaattgctgtgatgatgattatttcaataagaaggattggtatgacttcgtagagaatcctgacatcttacgcgtatatgctaacttaggtgagcgtgtacgccaatcattattaaagactgtacaattctgcgatgctatgcgtgatgcaggcattgtaggcgtactgacattagataatcaggatcttaatgggaactggtacgatttcggtgatttcgtacaagtagcaccaggctgcggagttcctattgtggattcatattactcattgctgatgcccatcctcactttgactagggcattggctgctgagtcccatatggatgctgatctcgcaaaaccacttattaagtgggatttgctgaaatatgattttacggaagagagactttgtctcttcgaccgttattttaaatattgggaccagacataccatcccaattgtattaactgtttggatgataggtgtatccttcattgtgcaaactttaatgtgttattttctactgtgtttccacctacaagttttggaccactagtaagaaaaatatttgtagatggtgttccttttgttgtttcaactggataccattttcgtgagttaggagtcgtacataatcaggatgtaaacttacatagctcgcgtctcagtttcaaggaacttttagtgtatgctgctgatccagctatgcatgcagcttctggcaatttattgctagataaacgcactacatgcttttcagtagctgcactaacaaacaatgttgcttttcaaactgtcaaacccggtaattttaataaagacttttatgactttgctgtgtctaaaggtttctttaaggaaggaagttctgttgaactaaaacacttcttctttgctcaggatggcaacgctgctatcagtgattatgactattatcgttataatctgccaacaatgtgtgatatcagacaactcctattcgtagttgaagttgttgataaatactttgattgttacgatggtggctgtattaatgccaaccaagtaatcgttaacaatctggataaatcagctggtttcccatttaataaatggggtaaggctagactttattatgactcaatgagttatgaggatcaagatgcacttttcgcgtatactaagcgtaatgtcatccctactataactcaaatgaatcttaagtatgccattagtgcaaagaatagagctcgcaccgtagctggtgtctctatctgtagtactatgacaaatagacagtttcatcagaaattattgaagtcaatagccgccactagaggagctactgtggtaattggaacaagcaagttttacggtggctggcataatatgttaaaaactgtttacagtgatgtagaaactccacaccttatgggttgggattatccaaaatgtgacagagccatgcctaacatgcttaggataatggcctctcttgttcttgctcgcaaacataacacttgctgtaacttatcacaccgtttctacaggttagctaacgagtgtgcgcaagtattaagtgagatggtcatgtgtggcggctcactatatgttaaaccaggtggaacatcatccggtgatgctacaactgcttatgctaatagtgtctttaacatttgtcaagctgttacagccaatgtaaatgcacttctttcaactgatggtaataagatagctgacaagtatgtccgcaatctacaacacaggctctatgagtgtctctatagaaatagggatgttgatcatgaattcgtggatgagttttacgcttacctgcgtaaacatttctccatgatgattctttctgatgatgccgttgtgtgctataacagtaactatgcggctcaaggtttagtagctagcattaagaactttaaggcagttctttattatcaaaataatgtgttcatgtctgaggcaaaatgttggactgagactgaccttactaaaggacctcacgaattttgctcacagcatacaatgctagttaaacaaggagatgattacgtgtacctgccttacccagatccatcaagaatattaggcgcaggctgttttgtcgatgatattgtcaaaacagatggtacacttatgattgaaaggttcgtgtcactggctattgatgcttacccacttacaaaacatcctaatcaggagtatgctgatgtctttcacttgtatttacaatacattagaaagttacatgatgagcttactggccacatgttggacatgtattccgtaatgctaactaatgataacacctcacggtactgggaacctgagttttatgaggctatgtacacaccacatacagtcttgcaggctgtaggtgcttgtgtattgtgcaattcacagacttcacttcgttgcggtgcctgtattaggagaccattcctatgttgcaagtgctgctatgaccatgtcatttcaacatcacacaaattagtgttgtctgttaatccctatgtttgcaatgccccaggttgtgatgtcactgatgtgacacaactgtatctaggaggtatgagctattattgcaagtcacataagcctcccattagttttccattatgtgctaatggtcaggtttttggtttatacaaaaacacatgtgtaggcagtgacaatgtcactgacttcaatgcgatagcaacatgtgattggactaatgctggcgattacatacttgccaacacttgtactgagagactcaagcttttcgcagcagaaacgctcaaagccactgaggaaacatttaagctgtcatatggtattgccactgtacgcgaagtactctctgacagagaattgcatctttcatgggaggttggaaaacctagaccaccattgaacagaaactatgtctttactggttaccgtgtaactaaaaatagtaaagtacagattggagagtacacctttgaaaaaggtgactatggtgatgctgttgtgtacagaggtactacgacatacaagttgaatgttggtgattactttgtgttgacatctcacactgtaatgccacttagtgcacctactctagtgccacaagagcactatgtgagaattactggcttgtacccaacactcaacatctcagatgagttttctagcaatgttgcaaattatcaaaaggtcggcatgcaaaagtactctacactccaaggaccacctggtactggtaagagtcattttgccatcggacttgctctctattacccatctgctcgcatagtgtatacggcatgctctcatgcagctgttgatgccctatgtgaaaaggcattaaaatatttgcccatagataaatgtagtagaatcatacctgcgcgtgcgcgcgtagagtgttttgataaattcaaagtgaattcaacactagaacagtatgttttctgcactgtaaatgcattgccagaaacaactgctgacattgtagtctttgatgaaatctctatggctactaattatgacttgagtgttgtcaatgctagacttcgtgcaaaacactacgtctatattggcgatcctgctcaattaccagccccccgcacattgctgactaaaggcacactagaaccagaatattttaattcagtgtgcagacttatgaaaacaataggtccagacatgttccttggaacttgtcgccgttgtcctgctgaaattgttgacactgtgagtgctttagtttatgacaataagctaaaagcacacaaggagaagtcagctcaatgcttcaaaatgttctacaaaggtgttattacacatgatgtttcatctgcaatcaacagacctcaaataggcgttgtaagagaatttcttacacgcaatcctgcttggagaaaagctgtttttatctcaccttataattcacagaacgctgtagcttcaaaaatcttaggattgcctacgcagactgttgattcatcacagggttctgaatatgactatgtcatattcacacaaactactgaaacagcacactcttgtaatgtcaaccgcttcaatgtggctatcacaagggcaaaaattggcattttgtgcataatgtctgatagagatctttatgacaaactgcaatttacaagtctagaaataccacgtcgcaatgtggctacattacaagcagaaaatgtaactggactttttaaggactgtagtaagatcattactggtcttcatcctacacaggcacctacacacctcagcgttgatataaagttcaagactgaaggattatgtgttgacataccaggcataccaaaggacatgacctaccgtagactcatctctatgatgggtttcaaaatgaattaccaagtcaatggttaccctaatatgtttatcacccgcgaagaagctattcgtcacgttcgtgcgtggattggctttgatgtagagggctgtcatgcaactagagatgctgtgggtactaacctacctctccagctaggattttctacaggtgttaacttagtagctgtaccgactggttatgttgacactgaaaataacacagaattcaccagagttaatgcaaaacctccaccaggtgaccagtttaaacatcttataccactcatgtataaaggcttgccctggaatgtagtgcgtattaagatagtacaaatgctcagtgatacactgaaaggattgtcagacagagtcgtgttcgtcctttgggcgcatggctttgagcttacatcaatgaagtactttgtcaagattggacctgaaagaacgtgttgtctgtgtgacaaacgtgcaacttgcttttctacttcatcagatacttatgcctgctggaatcattctgtgggttttgactatgtctataacccatttatgattgatgttcagcagtggggctttacgggtaaccttcagagtaaccatgaccaacattgccaggtacatggaaatgcacatgtggctagttgtgatgctatcatgactagatgtttagcagtccatgagtgctttgttaagcgcgttgattggtctgttgaataccctattataggagatgaactgagggttaattctgcttgcagaaaagtacaacacatggttgtgaagtctgcattgcttgctgataagtttccagttcttcatgacattggaaatccaaaggctatcaagtgtgtgcctcaggctgaagtagaatggaagttctacgatgctcagccatgtagtgacaaagcttacaaaatagaggaactcttctattcttatgctacacatcacgataaattcactgatggtgtttgtttgttttggaattgtaacgttgatcgttacccagccaatgcaattgtgtgtaggtttgacacaagagtcttgtcaaacttgaacttaccaggctgtgatggtggtagtttgtatgtgaataagcatgcattccacactccagctttcgataaaagtgcatttactaatttaaagcaattgcctttcttttactattctgatagtccttgtgagtctcatggcaaacaagtagtgtcggatattgattatgttccactcaaatctgctacgtgtattacacgatgcaatttaggtggtgctgtttgcagacaccatgcaaatgagtaccgacagtacttggatgcatataatatgatgatttctgctggatttagcctatggatttacaaacaatttgatacttataacctgtggaatacatttaccaggttacagagtttagaaaatgtggcttataatgttgttaataaaggacactttgatggacacgccggcgaagcacctgtttccatcattaataatgctgtttacacaaaggtagatggtattgatgtggagatctttgaaaataagacaacacttcctgttaatgttgcatttgagctttgggctaagcgtaacattaaaccagtgccagagattaagatactcaataatttgggtgttgatatcgctgctaatactgtaatctgggactacaaaagagaagccccagcacatgtgtctacaataggtgtctgcacaatgactgacattgccaagaaacctactgagagtgcttgttcttcacttactgtcttgtttgatggtagagtggaaggacaggtagacctttttagaaacgcccgtaatggtgttttaataacagaaggttcagtcaaaggtctaacaccttcaaagggaccagcacaagctagcgtcaatggagtcacattaattggagaatcagtaaaaacacagtttaactactttaagaaagtagacggcattattcaacagttgcctgaaacctactttactcagagcagagacttagaggattttaagcccagatcacaaatggaaactgactttctcgagctcgctatggatgaattcatacagcgatataagctcgagggctatgccttcgaacacatcgtttatggagatttcagtcatggacaacttggcggtcttcatttaatgataggcttagccaagcgctcacaagattcaccacttaaattagaggattttatccctatggacagcacagtgaaaaattacttcataacagatgcgcaaacaggttcatcaaaatgtgtgtgttctgtgattgatcttttacttgatgactttgtcgagataataaagtcacaagatttgtcagtgatttcaaaagtggtcaaggttacaattgactatgctgaaatttcattcatgctttggtgtaaggatggacatgttgaaaccttctacccaaaactacaagcaagtcaagcgtggcaaccaggtgttgcgatgcctaacttgtacaagatgcaaagaatgcttcttgaaaagtgtgaccttcagaattatggtgaaaatgctgttataccaaaaggaataatgatgaatgtcgcaaagtatactcaactgtgtcaatacttaaatacacttactttagctgtaccctacaacatgagagttattcactttggtgctggctctgataaaggagttgcaccaggtacagctgtgctcagacaatggttgccaactggcacactacttgtcgattcagatcttaatgacttcgtctccgacgcagattctactttaattggagactgtgcaacagtacatacggctaataaatgggaccttattattagcgatatgtatgaccctaggaccaaacatgtgacaaaagagaatgactctaaagaagggtttttcacttatctgtgtggatttataaagcaaaaactagccctgggtggttctatagctgtaaagataacagagcattcttggaatgctgacctttacaagcttatgggccatttctcatggtggacagcttttgttacaaatgtaaatgcatcatcatcggaagcatttttaattggggctaactatcttggcaagccgaaggaacaaattgatggctataccatgcatgctaactacattttctggaggaacacaaatcctatccagttgtcttcctattcactctttgacatgagcaaatttcctcttaaattaagaggaactgctgtaatgtctcttaaggagaatcaaatcaatgatatgatttattctcttctggaaaaaggtaggcttatcattagagaaaacaacagagttgtggtttcaagtgatattcttgttaacaactaaacgaacatgtttattttcttattatttcttactctcactagtggtagtgaccttgaccggtgcaccacttttgatgatgttcaagctcctaattacactcaacatacttcatctatgaggggggtttactatcctgatgaaatttttagatcagacactctttatttaactcaggatttatttcttccattttattctaatgttacagggtttcatactattaatcatacgtttgacaaccctgtcataccttttaaggatggtatttattttgctgccacagagaaatcaaatgttgtccgtggttgggtttttggttctaccatgaacaacaagtcacagtcggtgattattattaacaattctactaatgttgttatacgagcatgtaactttgaattgtgtgacaaccctttctttgctgtttctaaacccatgggtacacagacacatactatgatattcgataatgcatttaattgcactttcgagtacatatctgatgccttttcgcttgatgtttcagaaaagtcaggtaattttaaacacttacgagagtttgtgtttaaaaataaagatgggtttctctatgtttataagggctatcaacctatagatgtagttcgtgatctaccttctggttttaacactttgaaacctatttttaagttgcctcttggtattaacattacaaattttagagccattcttacagccttttcacctgctcaagacacttggggcacgtcagctgcagcctattttgttggctatttaaagccaactacatttatgctcaagtatgatgaaaatggtacaatcacagatgctgttgattgttctcaaaatccacttgctgaactcaaatgctctgttaagagctttgagattgacaaaggaatttaccagacctctaatttcagggttgttccctcaggagatgttgtgagattccctaatattacaaacttgtgtccttttggagaggtttttaatgctactaaattcccttctgtctatgcatgggagagaaaaaaaatttctaattgtgttgctgattactctgtgctctacaactcaacatttttttcaacctttaagtgctatggcgtttctgccactaagttgaatgatctttgcttctccaatgtctatgcagattcttttgtagtcaagggagatgatgtaagacaaatagcgccaggacaaactggtgttattgctgattataattataaattgccagatgatttcatgggttgtgtccttgcttggaatactaggaacattgatgctacttcaactggtaattataattataaatataggtatcttagacatggcaagcttaggccctttgagagagacatatctaatgtgcctttctcccctgatggcaaaccttgcaccccacctgctcttaattgttattggccattaaatgattatggtttttacaccactactggcattggctaccaaccttacagagttgtagtactttcttttgaacttttaaatgcaccggccacggtttgtggaccaaaattatccactgaccttattaagaaccagtgtgtcaattttaattttaatggactcactggtactggtgtgttaactccttcttcaaagagatttcaaccatttcaacaatttggccgtgatgtttctgatttcactgattccgttcgagatcctaaaacatctgaaatattagacatttcaccttgctcttttgggggtgtaagtgtaattacacctggaacaaatgcttcatctgaagttgctgttctatatcaagatgttaactgcactgatgtttctacagcaattcatgcagatcaactcacaccagcttggcgcatatattctactggaaacaatgtattccagactcaagcaggctgtcttataggagctgagcatgtcgacacttcttatgagtgcgacattcctattggagctggcatttgtgctagttaccatacagtttctttattacgtagtactagccaaaaatctattgtggcttatactatgtctttaggtgctgatagttcaattgcttactctaataacaccattgctatacctactaacttttcaattagcattactacagaagtaatgcctgtttctatggctaaaacctccgtagattgtaatatgtacatctgcggagattctactgaatgtgctaatttgcttctccaatatggtagcttttgcacacaactaaatcgtgcactctcaggtattgctgctgaacaggatcgcaacacacgtgaagtgttcgctcaagtcaaacaaatgtacaaaaccccaactttgaaatattttggtggttttaatttttcacaaatattacctgaccctctaaagccaactaagaggtcttttattgaggacttgctctttaataaggtgacactcgctgatgctggcttcatgaagcaatatggcgaatgcctaggtgatattaatgctagagatctcatttgtgcgcagaagttcaatggacttacagtgttgccacctctgctcactgatgatatgattgctgcctacactgctgctctagttagtggtactgccactgctggatggacatttggtgctggcgctgctcttcaaataccttttgctatgcaaatggcatataggttcaatggcattggagttacccaaaatgttctctatgagaaccaaaaacaaatcgccaaccaatttaacaaggcgattagtcaaattcaagaatcacttacaacaacatcaactgcattgggcaagctgcaagacgttgttaaccagaatgctcaagcattaaacacacttgttaaacaacttagctctaattttggtgcaatttcaagtgtgctaaatgatatcctttcgcgacttgataaagtcgaggcggaggtacaaattgacaggttaattacaggcagacttcaaagccttcaaacctatgtaacacaacaactaatcagggctgctgaaatcagggcttctgctaatcttgctgctactaaaatgtctgagtgtgttcttggacaatcaaaaagagttgacttttgtggaaagggctaccaccttatgtccttcccacaagcagccccgcatggtgttgtcttcctacatgtcacgtatgtgccatcccaggagaggaacttcaccacagcgccagcaatttgtcatgaaggcaaagcatacttccctcgtgaaggtgtttttgtgtttaatggcacttcttggtttattacacagaggaacttcttttctccacaaataattactacagacaatacatttgtctcaggaaattgtgatgtcgttattggcatcattaacaacacagtttatgatcctctgcaacctgagcttgactcattcaaagaagagctggacaagtacttcaaaaatcatacatcaccagatgttgatcttggcgacatttcaggcattaacgcttctgtcgtcaacattcaaaaagaaattgaccgcctcaatgaggtcgctaaaaatttaaatgaatcactcattgaccttcaagaattgggaaaatatgagcaatatattaaatggccttggtatgtttggctcggcttcattgctggactaattgccatcgtcatggttacaatcttgctttgttgcatgactagttgttgcagttgcctcaagggtgcatgctcttgtggttcttgctgcaagtttgatgaggatgactctgagccagttctcaagggtgtcaaattacattacacataaacgaacttatggatttgtttatgagattttttactcttggatcaattactgcacagccagtaaaaattgacaatgcttctcctgcaagtactgttcatgctacagcaacgataccgctacaagcctcactccctttcggatggcttgttattggcgttgcatttcttgctgtttttcagagcgctaccaaaataattgcgctcaataaaagatggcagctagccctttataagggcttccagttcatttgcaatttactgctgctatttgttaccatctattcacatcttttgcttgtcgctgcaggtatggaggcgcaatttttgtacctctatgccttgatatattttctacaatgcatcaacgcatgtagaattattatgagatgttggctttgttggaagtgcaaatcccagaacccattactttatgatgccaactactttgtttgctggcacacacataactatgactactgtataccatataacagtgtcacagatacaattgtcgttactgaaggtgacggcatttcaacaccaaaactcaaagaagactaccaaattggtggttattctgaggataggcactcaggtgttaaagactatgtcgttgtacatggctatttcaccgaagtttactaccagcttgagtctacacaaattactacagacactggtattgaaaatgctacattcttcatctttaacaagcttgttaaagacccaccgaatgtgcaaatacacacaatcgacggctcttcaggagttgctaatccagcaatggatcccatttatgatgagccgacgacgactactagcgtgcctttgtaagcacaagaaagtgagtacgaacttatgtactcattcgtttcggaagaaacaggtacgttaatagttaatagcgtacttctttttcttgctttcgtggtattcttgctagtcacactagccatccttactgcgcttcgattgtgtgcgtactgctgcaatattgttaacgtgagtttagtaaaaccaacggtttacgtctactcgcgtgttaaaaatctgaactcttctgaaggagttcctgatcttctggtctaaacgaactaactattattattattctgtttggaactttaacattgcttatcatggcagacaacggtactattaccgttgaggagcttaaacaactcctggaacaatggaacctagtaataggtttcctattcctagcctggattatgttactacaatttgcctattctaatcggaacaggtttttgtacataataaagcttgttttcctctggctcttgtggccagtaacacttgcttgttttgtgcttgctgctgtctacagaattaattgggtgactggcgggattgcgattgcaatggcttgtattgtaggcttgatgtggcttagctacttcgttgcttccttcaggctgtttgctcgtacccgctcaatgtggtcattcaacccagaaacaaacattcttctcaatgtgcctctccgggggacaattgtgaccagaccgctcatggaaagtgaacttgtcattggtgctgtgatcattcgtggtcacttgcgaatggccggacactccctagggcgctgtgacattaaggacctgccaaaagagatcactgtggctacatcacgaacgctttcttattacaaattaggagcgtcgcagcgtgtaggcactgattcaggttttgctgcatacaaccgctaccgtattggaaactataaattaaatacagaccacgccggtagcaacgacaatattgctttgctagtacagtaagtgacaacagatgtttcatcttgttgacttccaggttacaatagcagagatattgattatcattatgaggactttcaggattgctatttggaatcttgacgttataataagttcaatagtgagacaattatttaagcctctaactaagaagaattattcggagttagatgatgaagaacttatggagttagattatccataaaacgaacatgaaaattattctcttcctgacattgattgtatttacatcttgcgagctatatcactatcaggagtgtgttagaggtacgactgtactactaaaagaaccttgcccatcaggaacatacgagggcaattcaccatttcaccctcttgctgacaataaatttgcactaacttgcactagcacacactttgcttttgcttgtgctgacggtactcgacatacctatcagctgcgtgcaagatcagtttcaccaaaacttttcatcagacaagaggaggttcaacaagagctctactcgccactttttctcattgttgctgctctagtatttttaatactttgcttcaccattaagagaaagacagaatgaatgagctcactttaattgacttctatttgtgctttttagcctttctgctattccttgttttaataatgcttattatattttggttttcactcgaaatccaggatctagaagaaccttgtaccaaagtctaaacgaacatgaaacttctcattgttttgacttgtatttctctatgcagttgcatacgcactgtagtacagcgctgtgcatctaataaacctcatgtgcttgaagatccttgtaaggtacaacactaggggtaatacttatagcactgcttggctttgtgctctaggaaaggttttaccttttcatagatggcacactatggttcaaacatgcacacctaatgttactatcaactgtcaagatccagctggtggtgcgcttatagctaggtgttggtaccttcatgaaggtcaccaaactgctgcatttagagacgtacttgttgttttaaataaacgaacaaattaaaatgtctgataatggaccccaatcaaaccaacgtagtgccccccgcattacatttggtggacccacagattcaactgacaataaccagaatggaggacgcaatggggcaaggccaaaacagcgccgaccccaaggtttacccaataatactgcgtcttggttcacagctctcactcagcatggcaaggaggaacttagattccctcgaggccagggcgttccaatcaacaccaatagtggtccagatgaccaaattggctactaccgaagagctacccgacgagttcgtggtggtgacggcaaaatgaaagagctcagccccagatggtacttctattacctaggaactggcccagaagcttcacttccctacggcgctaacaaagaaggcatcgtatgggttgcaactgagggagccttgaatacacccaaagaccacattggcacccgcaatcctaataacaatgctgccaccgtgctacaacttcctcaaggaacaacattgccaaaaggcttctacgcagagggaagcagaggcggcagtcaagcctcttctcgctcctcatcacgtagtcgcggtaattcaagaaattcaactcctggcagcagtaggggaaattctcctgctcgaatggctagcggaggtggtgaaactgccctcgcgctattgctgctagacagattgaaccagcttgagagcaaagtttctggtaaaggccaacaacaacaaggccaaactgtcactaagaaatctgctgctgaggcatctaaaaagcctcgccaaaaacgtactgccacaaaacagtacaacgtcactcaagcatttgggagacgtggtccagaacaaacccaaggaaatttcggggaccaagacctaatcagacaaggaactgattacaaacattggccgcaaattgcacaatttgctccaagtgcctctgcattctttggaatgtcacgcattggcatggaagtcacaccttcgggaacatggctgacttatcatggagccattaaattggatgacaaagatccacaattcaaagacaacgtcatactgctgaacaagcacattgacgcatacaaaacattcccaccaacagagcctaaaaaggacaaaaagaaaaagactgatgaagctcagcctttgccgcagagacaaaagaagcagcccactgtgactcttcttcctgcggctgacatggatgatttctccagacaacttcaaaattccatgagtggagcttctgctgattcaactcaggcataaacactcatgatgaccacacaaggcagatgggctatgtaaacgttttcgcaattccgtttacgatacatagtctactcttgtgcagaatgaattctcgtaactaaacagcacaagtaggtttagttaactttaatctcacatagcaatctttaatcaatgtgtaacattagggaggacttgaaagagccaccacattttcatcgaggccacgcggagtacgatcgagggtacagtgaataatgctagggagagctgcctatatggaagagccctaatgtgtaaaattaattttagtagtgctatccccatgtgattttaatagcttcttaggagaatgacaaaaaaaaaaaaaaaaa

SEQ ID NO: 3 - DNA, 24 bp, Synthetic
gataatggaccccaatcaaaccaa

SEQ ID NO: 4 - DNA, 24 bp, Synthetic
ctgagttgaatcaggagaagctcc

SEQ ID NO: 5 - DNA, 22 bp, Synthetic
gaactggcccagaagcttcact

SEQ ID NO: 6 - DNA, 22 bp, Synthetic
ttggcctttaccagaaactttg